(19) United States
(12) Patent Application Publication
(10) Pub. No.: US 2016/0346313 A1
(43) Pub. Date: Dec. 1, 2016

(54) USES OF OLIGOURONATES IN CANCER TREATMENT

(71) Applicant: NORWEGIAN UNIVERSITY OF SCIENCE AND TECHNOLOGY, Trondheim (NO)

(72) Inventors: Catherine TAYLOR NORDGÅRD, Agdenes (NO); Kurt DRAGET, Trondheim (NO)

(21) Appl. No.: 15/107,424

(22) PCT Filed: Dec. 23, 2014
(86) PCT No.: PCT/EP2014/079178

§ 371 Date: Jun. 22, 2016

(30) Foreign Application Priority Data
Dec. 23, 2013 (GB) 1322958.8
Jun. 16, 2014 (GB) 1410695.9

(51) Int. Cl. A61K 31/716; A61K 45/06

(52) U.S. Cl. CPC A61K 31/716; A61K 45/06

(57) ABSTRACT

An oligouronate is used in the treatment or prevention of cancer as an anticancer agent. The oligouronate is effective against cancer itself, and as a drug delivery tool which improves or promotes the delivery of another anticancer agent, such as a chemotherapy drug or immunotherapeutic agent. Thus, the oligouronate is used in cancer prevention or treatment alone or in combination with one or more other anticancer agents.

This invention relates to the use of an oligouronate in the treatment or prevention of cancer, both as an anticancer agent, that is as an agent effective against cancer itself, and as a drug delivery tool, that is to improve or promote the delivery of a further or different anticancer agent, e.g., chemotherapy drug or immunotherapeutic agent. Thus, the invention relates to the use in cancer prevention or treatment of oligouronates both alone, and in combination with one or more additional or further anticancer agents. The invention also relates to methods of treating or preventing cancer using oligouronates, either alone or in combination with one or more further anticancer agents.

Cancer is a disease characterized by the loss of appropriate control of cell growth and proliferation. The American Cancer Society has estimated that there were in excess of 1.5 million new cases of cancer within the United Stated of America in 2010 and approximately 570,000 deaths that year estimated to be attributable to cancer. The World Health Organization has estimated that cancer was the leading cause of death globally in 2010, with the number of deaths caused by cancer growing to 12 million per year by 2030.

Whilst there are numerous therapies available, resistance to known anticancer drugs can be a problem in the successful treatment of cancer in patients. The limitations of clinical treatments such as chemotherapy have in the past been ascribed primarily to mechanisms that mediate drug resistance at the cellular level. For example, functional gene mutations or other alterations that affect the expression of proteins that influence the uptake, metabolism, and export of drugs from a cell are important determinants of drug resistance, as are epigenetic changes that can lead to transient drug release.

However, there is evidence that the extracellular matrix (ECM), i.e. the microenvironment, which surrounds cancer cells, also has a role to play in enabling these cells to develop resistance to, or be less amenable to, treatment with anticancer, including cytostatic/cytotoxic/chemotherapeutic or immunotherapeutic, agents. Although long viewed as a stable structure that plays a mainly supporting role in maintaining tissue morphology, the ECM is an essential part of the milieu of a cell, and is surprisingly dynamic and versatile and can influence fundamental aspects of cell biology. The ECM can directly or indirectly regulate cellular processes.

The ECM is the extracellular component of a multicellular organism. The ECM interacts with and provides structural and biochemical support to the surrounding cells. In animals the ECM comprises the interstitial fluid and basement membrane. Interstitial matrix is present between various animal cells (i.e., in the intercellular spaces). Gels of polysaccharides and fibrous proteins fill the interstitial space and act as a compression buffer against the stress placed on the ECM. Basement membranes are sheet-like depositions of ECM on which various epithelial cells rest.

Components of the ECM are produced intracellularly by resident cells and secreted into the ECM via exocytosis. Once secreted, they then aggregate with the existing matrix. The ECM is composed of a large collection of biochemically distinct components including proteins, glycoproteins, proteoglycans and polysaccharides, and resembles an interlocking mesh of fibrous proteins and glycosaminoglycans. Glycosaminoglycan (GAG) chains occupy large amounts of space and form porous hydrated gels, providing mechanical support to the tissue. GAGs are unbranched polysaccharide chains composed of repeating disaccharide units. One of the two sugars in the repeating disaccharide is always an amino sugar (N-acetylglucosamine or N-acetylgalactosamine), which in most cases is sulfated. The second sugar is usually an uronic acid (glucuronic or iduronic). As there are sulfate or carboxyl groups on most of their sugars, GAGs are highly negatively charged. Indeed, they are the most anionic molecules produced by animal cells. Four main groups of GAGs are distinguished according to their sugars, the type of linkage between the sugars, and the number and location of sulfate groups: (1) hyaluronan, (2) chondroitin sulfate and dermatan sulfate, (3) heparan sulfate, and (4) keratan sulfate.

Except for hyaluronan, all GAGs are found covalently attached to protein in the form of proteoglycans, which are made by most animal cells. The polypeptide chain, or core protein, of a proteoglycan is made on membrane-bound ribosomes and threaded into the lumen of the endoplasmic reticulum. The polysaccharide chains are mainly assembled on this core protein in the Golgi apparatus. First, a special link tetrasaccharide is attached to a serine side chain on the core protein to serve as a primer for polysaccharide growth; then, one sugar at a time is added by specific glycosyl transferases. While still in the Golgi apparatus, many of the polymerized sugars are covalently modified by a sequential and coordinated series of reactions. Epimerizations alter the configuration of the substituents around individual carbon atoms in the sugar molecule; sulfations increase the negative charge.

The ECM also contains fibrous proteins, of which collagens are the major component. The collagens are a family of fibrous proteins found in all multicellular animals. The primary feature of a typical collagen molecule is its long, stiff, triple-stranded helical structure, in which three collagen polypeptide chains (a chains), are wound around one another in a ropelike superhelix. Collagens are extremely rich in proline and glycine, both of which are important in the formation of the triple-stranded helix.

The ECM also contains elastin proteins. Many vertebrate tissues, such as skin, blood vessels, and lungs, need to be both strong and elastic in order to function. A network of elastic fibres in the extracellular matrix of these tissues gives them the required resilience so that they can recoil after transient stretch. The extracellular matrix also contains a number of noncollagen proteins that typically have multiple domains, each with specific binding sites for other matrix macromolecules and for receptors on the surface of cells. These proteins therefore contribute to both organizing the matrix and helping cells attach to it. The first of them to be well characterized was fibronectin, a large glycoprotein found in all vertebrates. Consistent with the important role of the ECM in cell biology, multiple regulatory mechanisms have evolved to ensure correct control of its functions. Disruption to such control mechanisms deregulates and disorganises the ECM, which can lead to abnormal cellular behaviour of cells residing therein. Indeed, abnormal ECM dynamics are apparent in the context of cancerous cells. Thus, altered ECM can be a hallmark of cancer.

In addition to changes in biochemical properties, the physical properties of ECM in a cancerous environment can be altered. For example, type I collagen may become thickened and oriented, e.g. linearized, resulting in ECM which is stiffer than normal. This can be a characteristic of certain cancers, for example certain breast cancers exhibit increased tissue stiffness. ECM remodelling enzymes are also often deregulated in cancerous tissues.

Alterations, such as thickening or stiffening of the ECM, or changes in the amount and/or composition of the ECM, can create a barrier to anticancer agents such as chemotherapeutic agents and immunotherapeutic agents, which must reach the target cancer cell in order to be effective. The delivery of such drugs or agents to cancer cells can be impeded by an altered ECM, and therefore an altered ECM can affect the sensitivity of cancer cells to drug treatment.

Thus in addition to the continuing need for new anticancer therapies there is also a need for an effective means of enabling, facilitating or enhancing the delivery of anticancer agents, including chemotherapeutic and immunotherapeutic drugs and agents, to target cancer cells, particularly in the context of cancer cells with an altered ECM.

The present inventors have surprisingly found that oligouronates can be used for such a purpose. In particular, it has surprisingly been found that oligouronates may alter the physical properties of ECM, rendering it less viscous, or gel-like, in structure (or less solid-like in behaviour). In other words, oligouronates may disrupt, or break down, the structure of the ECM, thereby facilitating access or passage of molecules to or through the ECM, in particular through the ECM to underlying cancer cells. In particular, the oligouronates may restructure or re-model the ECM, rendering the structure more “open”, with hole-like openings becoming apparent. Without wishing to be bound by theory, it is presently believed that the oligouronates, and alginate oligomers in particular, do not degrade the components of the ECM as such, but rather alter the structure of the ECM as a whole, such that it becomes more open, or more porous. Whilst oligouronates have previously been proposed to reduce the viscosity of mucus, including in particular hyperviscous mucus such as occurs in sufferers of cystic fibrosis and other respiratory diseases (see WO 2007/039754 and WO 2008/125828), mucus is an entirely different material to ECM and it could not have been predicted from this that oligouronates would also have an effect on the structure of ECM.

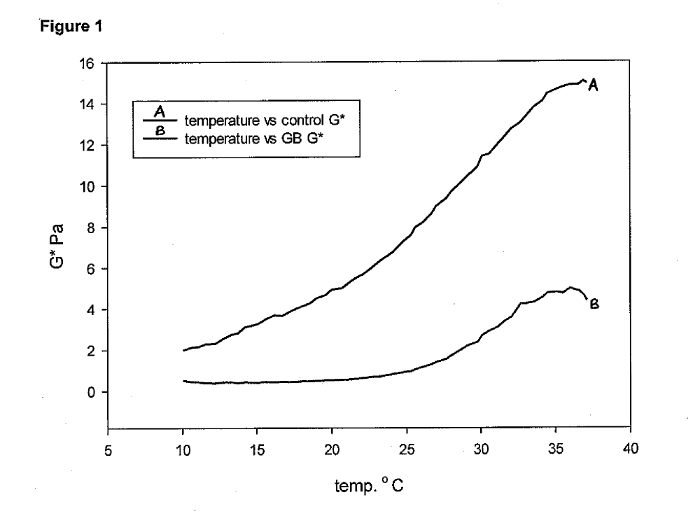

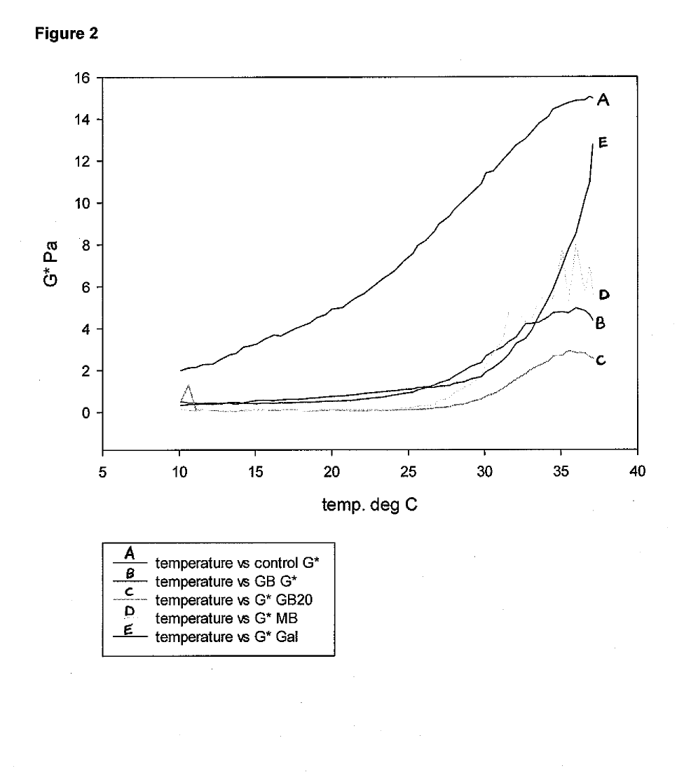

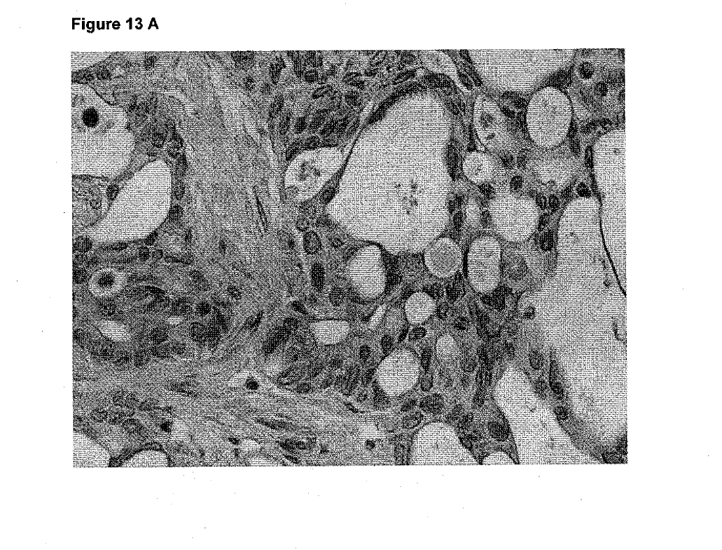

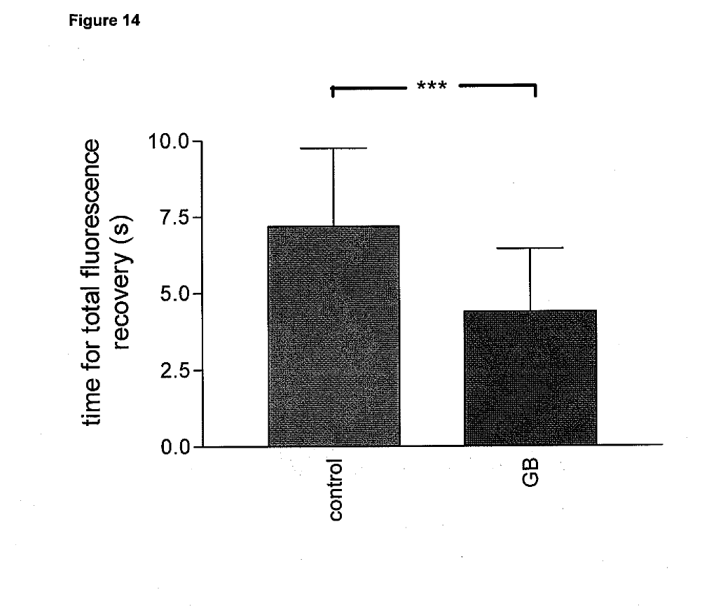

The results presented in the Examples below (see e.g. FIG. 12) demonstrate that a treatment with an oligouronate can result in changes in a structure of ECM. For example, it appears that oligouronates can cause less tight ECM and induce “openings” or “holes” in the ECM, which can improve the accessibility of anticancer agents to cancer cells within the ECM In addition, the results presented in the present Examples (see in particular FIGS. 1 and 2) demonstrate that various oligouronates, and alginate oligomers in particular, especially those with a high guluronate content (“high G”), can decrease the viscosity or gel strength of Matrigel™ (BD Biosciences). Matrigel™ is a solubilised basement membrane preparation extracted from a mouse sarcoma which is rich in extracellular ECM proteins including laminin, collagen IV, heparin sulphate proteoglycans and entactin/nidogen. Matrigel is thus an ECM preparation. In addition, Example 8 and FIG. 14 demonstrate that treatment of Matrigel with a “a high G” alginate oligomer facilitates faster diffusion of an immunoglobulin molecule (IgG) through the Matrigel as compared to a control Matrigel without the alginate oligomer.

Based on such results, it is proposed that oligouronates may make it easier for anticancer agents to penetrate or traverse the ECM in the context of cancer treatment, including in particular an altered ECM such as may occur in cancer. Thus, it is proposed that oligouronates make the ECM, and hence cancer cells within it, more accessible to external agents such as anticancer agents, including macromolecules such as proteins (e.g. therapeutic antibodies). This has been borne out by animal studies, also reported in the Examples below, which show that co-administration of an oligouronate with a chemotherapeutic agent enhanced the anti-tumour response of the animal, and in particular that a greater reduction in tumour weight could be achieved, as compared to the chemotherapeutic agent alone.

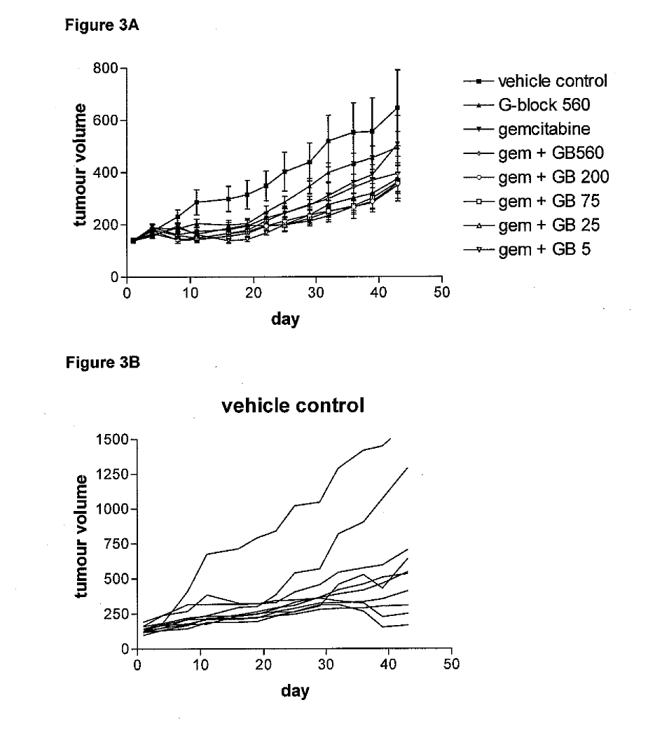

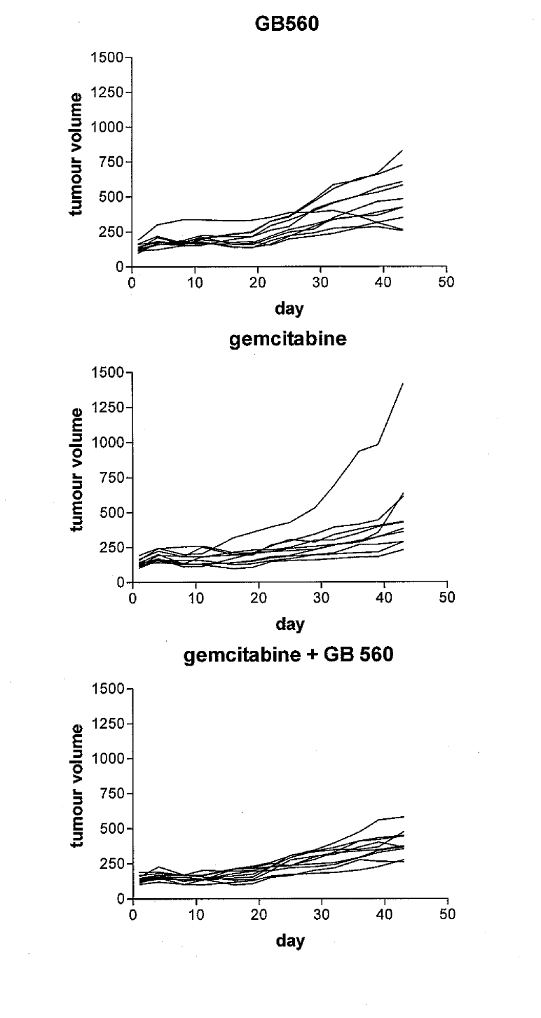

Further and surprisingly, in such animal studies the control experiments using the oligouronate alone show that it has an unexpected anticancer effect on its own, reducing the extent of tumour growth. In particular, as can be seen from FIGS. 3 and 4 and Examples 2 and 3 below, the oligouronate administered alone showed an unexpected beneficial effect in reducing tumour size in the in vivo animal model of pancreatic cancer. Such an effect was entirely unpredicted and very surprising, as it has not been previously demonstrated or in any way suggested that oligouronates themselves may have anticancer properties. This accordingly leads to the proposal that an oligouronate may be an effective anticancer agent in its own right, and hence that it may be used alone to treat or prevent cancer, or in combination with other anticancer agents, both as a combination therapy or to facilitate or enhance the delivery of another anticancer agent.

In a first aspect, the present invention therefore provides an oligouronate for use as an anticancer agent, i.e. for use in the treatment or prevention of cancer, or alternatively put the use of an oligouronate for treating or preventing cancer.

Alternatively viewed, this aspect of the present invention provides use of an oligouronate in the manufacture of an anticancer therapeutic product (i.e. a preparation or medicament, for example a pharmaceutical composition, formulation, combined product or kit) (or alternatively put, for the manufacture of a medicament for use as an anticancer agent or in the treatment or prevention of cancer).

As noted above, in the treatment or prevention of cancer the oligouronate may be used alone or optionally in combination with another (i.e. further or additional or second) anticancer agent.

The oligouronate, either alone or in combination with another anticancer drug, may be used according to the present invention in any method of treatment or prevention of cancer in a subject.

Accordingly, in a further aspect, the present invention also provides a method of treating or preventing cancer in a subject, which method comprises administering an oligouronate, optionally together with another anticancer agent, to said subject. Particularly, in this aspect the method comprises administering an effective amount of said oligouronate and the optional further anticancer agent.

Accordingly, further embodiments of the present invention provide an oligouronate together with an anticancer agent for use in treating or preventing cancer, or alternatively put, an oligouronate for use together with an anticancer agent for treating or preventing cancer.

As described above, according to the present invention oligouronates have particular utility in promoting, facilitating, increasing, improving or enhancing the delivery, or uptake, of an anticancer agent (specifically the uptake of the agent across the ECM). In other words the oligouronate may be used as a drug delivery agent for an anticancer agent.

Accordingly in a still further aspect the present invention provides an oligouronate for use in enhancing the delivery of an anticancer agent.

More particularly in this aspect the oligouronate is used to enhance the delivery of an anticancer agent in the treatment and prevention of cancer.

Alternatively viewed, this aspect of the present invention provides the use of an oligouronate in the manufacture of a medicament further comprising an anticancer drug to enhance the delivery of the anticancer agent.

As noted above, the medicament may be a combined preparation, composition or kit etc. and it is not necessary in any of the aspects of the invention for the oligouronate and other anticancer agent to be co-formulated in a single composition—they may be separately formulated and may be administered separately, including sequentially or simultaneously.

Accordingly, the invention also provides a kit comprising an oligouronate and an (or a further) anticancer agent for using in the treatment or prevention of cancer and/or for enhancing delivery of the anticancer agent to a subject in need thereof.

More particularly, the invention provides a product (particularly a pharmaceutical product) comprising an oligouronate and an (or further or second) anticancer agent as a combined preparation for separate, sequential or simultaneous use in the treatment or prevention of cancer, and/or for enhancing delivery of the anticancer agent to a subject in need thereof.

In a further aspect the invention provides a method for enhancing the delivery of an anticancer agent, said method comprising co-administering said anticancer agent with an oligouronate to a subject in need of said anticancer agent.

In these aspects of the invention presented above the oligouronate is preferably an alginate oligomer, and more particularly a “high G” alginate oligomer, that is an alginate oligomer comprising at least 70% guluronate (G) residues. Specifically the preferred oligouronate may be defined as containing (i.e. having or consisting of) up to 100 monomer residues, at least 70% of which are guluronate (G) residues.

As described above, this aspect has particular application in the treatment of cancers where the cancer has an altered ECM, and more particularly where the altered ECM exhibits reduced penetrability to anticancer agents i.e. is less penetrable or less permeable or accessible, or more resistant, to anticancer agents.

An altered ECM is an ECM which is different to, or modified as compared to, an ECM which occurs in a healthy tissue. In particular, in the context of cancer the ECM is altered in the cancerous tissue or tumour as compared to the corresponding non-cancerous or healthy tissue, i.e. in the tissue in which, or from which, the cancer arises. An altered ECM may accordingly alternatively be defined as abnormal or aberrant or modified. In particular embodiments the altered ECM may be disorganised and its formation and/or behaviour may be dysregulated. The ECM dynamics may be deregulated and in particular ECM remodelling may be deregulated. The ECM may be altered with respect to amount, composition and/or topography. For example, there may be increased or excess ECM production, or reduced ECM turnover. There may be increased production of certain ECM components, for example certain proteins e.g. increased collagen deposition. The nature, and hence physical and/or biochemical properties, of one or more constituent proteins or polysaccharide components may be altered. These changes may lead to different architecture in the altered ECM or different physical properties.

For example, there may be changes in the orientation of the protein components, particularly framework components of the ECM. Collagen fibres may become thickened and/or linearized. There may be increased cross-linking of collagen and/or other ECM proteins or components. Such alterations may result in an ECM which is thicker and/or stiffer and/or denser than an unaltered or normal ECM. In addition to such physical changes, the changes in biochemical properties of the altered ECM may lead it to have altered interactions with cells, including stromal cells, stem cells and immune cells, as well as the cells of the underlying tissue. Such effects have been postulated to play a role in cancer development and progression. The alterations in the properties of the ECM, most notably the physical or structural changes can lead to the altered ECM being impenetrable or less penetrable to administered anticancer agents.

Although, as noted above, many if not most cancers have an altered ECM, the altered ECMs may vary as between different cancers e.g. in different tissues or different cancer types. Certain cancers exhibit an altered ECM which presents a greater barrier to drug delivery, for example as compared not only to normal ECM but also altered ECM in other cancers. Such cancers are accordingly more resistant to anticancer agent therapy. Such cancers, which have an altered ECM which is less penetrable to anticancer agents (or to the specific anticancer agents of choice) are of particular interest in the present invention. Such cancers include for example those with a stiffer and/or thicker and/or denser ECM (e.g. with a more ordered or tighter or more cross-linked network of proteins, for example those with increased cross-linking of collagen and/or other proteins).

The term “oligouronate” denotes molecules being oligomers containing uoronic acid residues, more particularly mannuronic and/or guluronic residues or galacturonic acid residues. An oligomer is broadly defined herein to mean a molecule of less than 200, more particularly less than 195 or 190 residues or monomer units. Alternatively defined, an oligomer has a molecular weight of less than 35,000 Da, more particularly less than 30,000, 25,000 or 20,000 Da.

Oligouronates preferably are composed entirely or substantially entirely of uronate residues as monomer units. In other words, although they may be chemically modified (see further below), it is preferred that the backbone of the oligomer is composed of uronic acid residues. They are preferably linear molecules, and in particular they are preferably non-cyclic.

Oligouronates may readily be obtained from natural sources since many natural polysaccharides contain uronic acid residues such as guluronic, mannuronic and galacturonic acid residues. Such polysaccharides may be cleaved or hydrolysed to generate oligouronate molecules.

Polysaccharide to oligosaccharide cleavage to produce oligouronates useable according to the present invention may be performed using conventional polysaccharide lysis techniques such as enzymatic or acid hydrolysis. Oligouronates may then be separated from the polysaccharide breakdown products chromatographically using an ion exchange resin or by fractionated precipitation or solubilisation. Techniques for oligouronate preparation are known in the art and described in the literature.

Examples of polysaccharides containing uronates include naturally occurring polysaccharides (such as xanthan, pectin, alginates, hyaluronan, heparin and chondroitin sulphate), and chemically modified polysaccharides, including but not limited to polysaccharides modified to add charged groups (such as carboxylated or carboxymethylated glycans), and polysaccharides modified to alter flexibility (e.g. by periodate oxidation). Suitable polysaccharides are discussed for example in “Handbook of Hydrocolloids”, Ed. Phillips and Williams, CRC, Boca Raton, Fla., USA, 2000. The use of alginates to generate alginate oligomers is however preferred as these naturally occur as block copolymers of mannuronic (M) and guluronic (G) acids; such polymers may be hydrolysed to release oligomers corresponding to these “blocks” and G-block or M-block oligomers, or indeed combined “MG”-block oligomers, can readily be isolated or separated from such alginate hydrolysis mixtures.

Oligomers of desired G and/or M content or G-block or M-block composition may be obtained by selecting appropriate alginate starting materials. For example certain alginates have higher G or M content or have more or longer stretches (blocks) of G (or M). Further appropriate hydrolysis and/or fractionation or separation conditions may be selected to favour production of the desired alginate oligomer (oligouronate).

Where alginates are used as the starting material for preparation of the oligouronate, the guluronic acid content may if desired be increased by epimerization with mannouronan C-5 epimerases.

Oligoguluronic acids suitable for use according to the invention may conveniently be produced by acid hydrolysis of alginic acid from algal sources, e.g Laminaria hyperborea, dissolution at neutral pH, addition of mineral acid to reduce the pH to 3.4 to precipitate the oligoguluronic acid, washing with weak acid, resuspension at neutral pH and freeze drying. Such processing results in a mixed preparation of oligoguluronates which vary in size, and possibly also in G-content. Further fractionation and or separation steps may be employed to obtain a preparation with a desired (e.g more homogenous or uniform) size range and/or G-content.

Oligogalacturonates may be obtained by hydrolyis or digestion of pectin containing materials, again according to procedures known in the art.

As noted above the oligouronates of use in the present invention may preferably contain or comprise guluronate, mannuronate and/or galacturonate residues. Oligomers containing, comprising or consisting of guluronate and/or mannuronate residues are particularly preferred and may collectively be referred to as alginate oligomers, since they may conveniently be derived from alginates. Alginates are linear polymers of (1-4) linked β-D-mannuronic acid (M) and/or its C-5 epimer α-L-guluronic acid (G). The primary structure of alginates can vary greatly. The M and G residues can be organised as homopolymeric blocks of contiguous M or G residues, as blocks of alternating M and G residues and single M or G residues can be found interspacing these block structures. An alginate molecule can comprise some or all of these structures and such structures might not be uniformly distributed throughout the polymer. In the extreme, there exists a homopolymer of guluronic acid (polyguluronate) or a homopolymer of mannuronic acid (polymannuronate).

Alginates have been isolated from marine brown algae (e.g. certain species of Durvillea, Lessonia and Laminaria) and bacteria such as Pseudomonas aeruginosa and Azotobacter vinelandii. Other pseudomonads (e.g. Pseudomonas fluorescens, Pseudomonas putida, and Pseudomonas mendocina) retain the genetic capacity to produce alginates but in the wild they do not produce detectable levels of alginate. By mutation these non-producing pseudomonads can be induced to produce stably large quantities of alginate.

Alginate is synthesised as polymannuronate and G residues are formed by the action of epimerases (specifically C-5 epimerases) on the M residues in the polymer. In the case of alginates extracted from algae, the G residues are predominantly organised as G blocks because the enzymes involved in alginate biosynthesis in algae preferentially introduce the G neighbouring another G, thus converting stretches of M residues into G-blocks. Elucidation of these biosynthetic systems has allowed the production of alginates with specific primary structures (WO 94/09124, Gimmestad, M et al, Journal of Bacteriology, 2003, Vol 185(12) 3515-3523 and WO 2004/011628).

Alginates are typically isolated from natural sources as large high molecular weight polymers (e.g. an average molecular weight in the range 300,000 to 500,000 Daltons. Such large alginate polymers may be degraded, or broken down, e.g. by chemical or enzymatic hydrolysis to produce alginate structures of lower molecular weight. Alginates that are used industrially typically have an average molecular weight in the range of 100,000 to 300,000 Daltons (i.e. such alginates are still considered to be large polymers) although alginates of an average molecular weight of approximately 35,000 Daltons have been used in pharmaceuticals.

As noted above, alginates typically occur as polymers of an average molecular weight of at least 35,000 Daltons i.e. approximately 175 to 190 monomer residues, although typically much higher and an alginate oligomer according to the present invention may be defined as a material obtained by fractionation (i.e. size reduction) of an alginate polymer, commonly a naturally occurring alginate. An alginate oligomer can be considered to be an alginate of an average molecular weight of less than 35,000 Daltons (i.e. less than approximately 190 or less than 175 monomer residues), in particular an alginate of an average molecular weight of less than 30,000 Daltons (i.e. less than approximately 175 or less than 150 monomer residues) more particularly an average molecular weight of less than 25,000 or 20,000 Daltons (i.e. less than approximately 135 or 125 monomer residues or less than approximately 110 or 100 monomer residues).

An oligomer generally comprises 2 or more units or residues and an oligouronate, or more particularly an alginate oligomer, for use according to the invention may as noted above contain up to 200 monomer residues, preferably up to 195 or 190 residues, more particularly up to 180, 170, 150, 120, 100, 95, 90, 85, 80, 75, 70, 65, 60, 55 or 50 residues. Typically it will contain 2 to 100 monomer residues, preferably 2 to 75, 2 to 70, 2 to 60, 2 to 50, 2 to 45, 2 to 40, 2 to 35, 2 to 32 or 2 to 30 residues. Other preferred ranges include any one of 3, 4, 5 or 6 to any one of 100, 75, 60, 50, 45, 40, 35, 32, 30, 27, 25, 22 or 20. Representative preferred ranges according to the present invention include particularly, 2 to 100, 3 to 100, 4 to 100, 5 to 100, 2 to 75, 3 to 75, 4 to 75, 5 to 75, 2 to 50, 3 to 50, 4 to 50, 5 to 50, 2 to 35, 3 to 35, 4 to 35 and 5 to 35. Alternatively defined an oligouronate for use according to the invention will typically have an average molecular weight of 350 to 20,000 Daltons, preferably 350 to 15,000 Daltons, preferably 350 to 10,000 Daltons and more preferably 350 to 8000 Daltons, 350 to 7000 Daltons, or 350 to 6,000 Daltons.

Alternatively put, the oligouronate (e.g. alginate oligomer) may have a degree of polymerisation (DP), or a number average degree of polymerisation (DPn) of 2 to 100, 2 to 75, 2 to 70, 2 to 50, 2 to 45, 2 to 40, 2 to 35, 2 to 32 or 2 to 30. More particularly the DP or DPn range can be any one of 3, 4, 5 or 6 to any one of 100, 75, 60, 50, 45, 40, 35, 32, 30, 27, 25, 23 or 20.

An alginate oligomer will, as noted above, contain (or comprise) guluronate or guluronic acid (G) and/or mannuronate or mannuronic acid (M) residues or units. An alginate oligomer according to the invention will preferably be composed solely, or substantially solely (i.e. consist essentially of) uronate/uronic acid residues, more particularly solely or substantially solely of G and/or M residues. Alternatively expressed, in the alginate oligomer of use in the present invention, at least 80%, more particularly at least 85, 90, 95 or 99% of the monomer residues may be uronate/uronic acid residues, or, more particularly G and/or M residues. In other words, preferably the alginate oligomer will not comprise other residues or units (e.g. other saccharide residues, or more particularly other uronic acid/uronate residues).

The alginate oligomer is preferably a linear oligomer.

Although this is less preferred, analogously an oligogalacturonate may comprise or consist solely or substantially solely of galacturonic acid residues i.e. at least 80%, more particularly at least 85, 90, 95 or 99% of the monomer residues are galacturonic acid residues.

More particularly, in a preferred embodiment at least 30% of the monomer residues of the alginate oligomer are G residues (i.e. guluronate or guluronic acid). In other words the alginate oligomer will contain at least 30% guluronate (or guluronic acid) residues, more particularly at least 40, 50, 60 or 70% G (guluronate) residues. Thus, a representative alginate oligomer for use according to the present invention may contain at least 70% G residues (i.e. at least 70% of the monomer residues of the alginate oligomer will be G residues).

Preferably at least 60%, more particularly at least 70% or 75%, even more particularly at least 80, 85, 90, 92, 95 or 99% of the monomer residues of an oligouronate, or alginate oligomer, are guluronate. In one embodiment the alginate oligomer may be a homooligomer of G, or 100% G).

It will be understood from the above that the term “oligoguluronate” as used herein does not imply that all the monomer residues of the oligomer are guluronic acid residues, but rather that the oligomer contains guluronic acid residues. The terms oligomannuronate and oligogalacturonate are to be interpreted analogously.

In a representative preferred embodiment, the oligouronate, or more particularly the alginate oligomer, has up to 100, 90, 85, 80, 75, 70, 65, 60, 55, 50, 45, 40, 35 or 30 monomer residues (or an analogous, corresponding, or equivalent DP or DPn as defined above) and comprises at least 70%, 75%, 80%, 82%, 85% or 90% G residues.

In a further preferred embodiment, the above described alginates of the invention have a primary structure wherein the majority of the G residues are in so called G-blocks. Preferably at least 50%, more preferably at least 70 or 75%, and most preferably at least 80, 85, 90 or 95% of the G residues are in G-blocks. A G block is a contiguous sequence of at least two G residues, preferably at least 3 contiguous G residues, more preferably at least 4 or 5 contiguous G residues, most preferably at least 7 contiguous G residues.

In particular at least 80, 85 or 90% of the G residues are linked 1-4 to another G residue. More particularly at least 95%, more preferably at least 98%, and most preferably at least 99% of the G residues of the alginate are linked 1-4 to another G residue.

The oligouronate of use in the invention is preferably a 3- to 35-mer, more preferably a 3- to 30- or 3- to 28-mer, in particular a 4- to 30- mer or 4- to 25-mer, especially a 6- to 30-, 6- to 25- or 6- to 22-mer e.g. a 8- to 30-, 8-to 25- or 8- to 20-mer, for example a 10- to 20- or 10- to 18-, 10- to 17- or 10- to 15-mer, e.g. having a molecular weight in the range 350 to 6400 Daltons or 350 to 6000 Daltons, preferably 550 to 5500 Daltons, preferably 750 to 5000 Daltons, and especially 750 to 4500 Daltons.

It may be a single compound or it may be a mixture of compounds, e.g. of a range of degrees of polymerization. As noted above hydrolysis of a polysaccharide typically results in a mixture of oligomers with a range of sizes. The size range may be reduced by separation or fractionation to produce a more uniform or less variable mixture. As also noted above, the monomeric residues in the oligouronate, may be the same or different and not all need carry electrically charged groups although it is preferred that the majority (e.g. at least 60%, preferably at least 80% more preferably at least 90%) do. It is preferred that a substantial majority, e.g. at least 80%, more preferably at least 90% of the charged groups have the same polarity. In the oligouronate, the ratio of hydroxyl groups to charged groups is preferably at least 2:1, more especially at least 3:1.

The oligouronate of the invention may have a degree of polymerisation (DP), or a number average degree of polymerisation (DPₙ), of 3-28, 4-25, 6-22, 8-20 or 10-15, or 5 to 18 or 7 to 15 or 8 to 12, especially 10.

The oligouronate of the invention may have a degree of polymerisation (DP), or a number average degree of polymerisation (DPₙ), of 8-50, 8-40, 8-35, 8-30, 8-28, 8-25, 8-22, 8-20, 8-18, 8-16 or 8-14.

The oligouronate of the invention may have a degree of polymerisation (DP), or a number average degree of polymerisation (DPₙ), of 9-50, 9-40, 9-35, 9-30, 9-28, 9-25, 9-22, 9-20, 9-18, 9-16 or 9-14.

The oligouronate of the invention may have a degree of polymerisation (DP), or a number average degree of polymerisation (DPₙ), of 10-50, 10-40, 10-35, 10-30, 10-28, 10-25, 10-22, 10-20, 10-18, 10-16 or 10-14.

The oligouronate of the invention may have a degree of polymerisation (DP), or a number average degree of polymerisation (DPₙ), of 12-50, 12-40, 12-35, 12-30, 12-28, 12-25, 12-22, 12-20, 12-18, 12-16 or 12-14.

The oligouronate of the invention may have a degree of polymerisation (DP), or a number average degree of polymerisation (DPₙ), of 15-50, 15-40, 15-35, 15-30, 15-28, 15-25, 15-22, 15-20, 15-18 or 15-16.

The oligouronate of the invention may have a degree of polymerisation (DP), or a number average degree of polymerisation (DPₙ), of 18-50, 18-40, 18-35, 18-30, 18-28, 18-25, 18-22 or 18-20.

Preferably the oligouronate of the invention is substantially free, preferably essentially free, of oligouronates having a degree of polymerisation outside of the ranges disclosed herein. This may be expressed in terms of the molecular weight distribution of the oligouronate of the invention, e.g. the percentage of each mole of the oligouronate being used in accordance with the invention which has a DP outside the relevant range. The molecular weight distribution is preferably such that no more than 10%, preferably no more than 9, 8, 7, 6, 5, 4, 3, 2, or 1% mole has a DP of three, two or one higher than the relevant upper limit for DPₙ. Likewise it is preferred that no more than 10%, preferably no more than 9, 8, 7, 6, 5, 4, 3, 2, or 1% mole has a DP below a number three, two or one smaller than the relevant lower limit for DPₙ.

As described in WO2008/125828 it is preferred further to fractionate or separate an oligouronate mixture obtained by hydrolysis of a polysaccharide to remove the so-called high molecular weight tail, namely to remove the higher molecular weight components of the mixture and reduce its homogeneity. The molecular weight distribution is preferably such that no more than 5% mole has a DP of two higher than the relevant upper limit for DPₙ. Likewise it is preferred that no more than 5% mole has a DP below a number two smaller than the relevant lower limit for DPₙ.

Suitable alginate oligomers are described in WO2007/039754, WO2007/039760, and WO 2008/125828, the disclosures of which are explicitly incorporated by reference herein in their entirety.

Representative suitable oligouronates have a DPₙ in the range 5 to 30, a guluronate/galacturonate fraction (F_{G}) of at least 0.80, a mannuronate fraction (F_{M}) of no more than 0.20, and at least 95 mole% of DP no more than 25.

Further suitable oligouronates have a number average degree of polymerization in the range 7 to 15 (preferably 8 to 12), a guluronate/galacturonate fraction (F_{G}) of at least 0.85 (preferably at least 0.90), a mannuronate fraction (F_{M}) of no more than 0.15 (preferably no more than 0.10), and having at least 95% mole with a degree of polymerization less than 17 (preferably less than 14).

Further suitable oligouronates have a number average degree of polymerization in the range 5 to 18 (especially 7 to 15), a guluronate/galacturonate fraction (F_{G}) of at least 0.80 (preferably at least 0.85, especially at least 0.92), a mannuronate fraction (F_{M}) of no more than 0.20 (preferably no more than 0.15, especially no more than 0.08), and having at least 95% mole with a degree of polymerization less than 20 (preferably less than 17).

Further suitable oligouronates have a number average degree of polymerization in the range 5 to 18, a guluronate/galacturonate fraction (F_{G}) of at least 0.92, a mannuronate fraction (F_{M}) of no more than 0.08, and having at least 95% mole with a degree of polymerization less than 20.

Further suitable oligouronates have a number average degree of polymerization in the range 5 to 18 (preferably 7 to 15, more preferably 8 to 12, especially about 10), a guluronate/galacturonate fraction (F_{G}) of at least 0.80 (preferably at least 0.85, more preferably at least 0.90, especially at least 0.92, most especially at least 0.95), a mannuronate fraction (F_{M}) of no more than 0.20 (preferably no more than 0.15, more preferably no more than 0.10, especially no more than 0.08, most especially no more than 0.05), and having at least 95% mole with a degree of polymerization less than 20 (preferably less than 17, more preferably less than 14).

Further suitable oligouronates have a number average degree of polymerization in the range 7 to 15 (preferably 8 to 12), a guluronate/galacturonate fraction (F_{G}) of at least 0.92 (preferably at least 0.95), a mannuronate fraction (F_{M}) of no more than 0.08 (preferably no more than 0.05), and having at least 95% mole with a degree of polymerization less than 17 (preferably less than 14).

Further suitable oligouronates have a number average degree of polymerization in the range 5 to 18, a guluronate/galacturonate fraction (F_{G}) of at least 0.80, a mannuronate fraction (F_{M}) of no more than 0.20, and having at least 95% mole with a degree of polymerization less than 20.

Further suitable oligouronates have a number average degree of polymerization in the range 7 to 15, a guluronate/galacturonate fraction (F_{G}) of at least 0.85, a mannuronate fraction (F_{M}) of no more than 0.15, and having at least 95% mole with a degree of polymerization less than 17.

Further suitable oligouronates have a number average degree of polymerization in the range 7 to 15, a guluronate/galacturonate fraction (F_{G}) of at least 0.92, a mannuronate fraction (F_{M}) of no more than 0.08, and having at least 95% mole with a degree of polymerization less than 17.

It will thus be seen that a particular class of oligouronates favoured according to the present invention is alginate oligomers defined as so-called “high G” or “G-block” oligomers i.e. having a high content of G residues or G-blocks (e.g. wherein at least 70% of the monomer residues are G, preferably arranged in G-blocks). However, other types of alginate oligomer may also be used, including in particular “high M” or “M-block” oligomers or MG-block oligomers, as described further below. Accordingly, it is alginate oligomers with high proportions of a single monomer type, and with said monomers of this type being present predominantly in contiguous sequences of that monomer type, that represent oligomers that are particularly preferred, e.g. oligomers wherein at least 70% of the monomer residues in the oligomer are G residues linked 1-4 to another G-residue, or more preferably at least 75%, and most preferably at least 80, 85, 90, 92, 93, 94, 95, 96, 97, 98, 99% of the monomers residues of the oligomer are G residues linked 1-4 to another G residue. This 1-4 linkage of two G residues can be alternatively expressed as a guluronic unit bound to an adjacent guluronic unit.

In a further embodiment at least, or more particularly more than, 50% of the monomer residues of the alginate oligomer may be M residues (i.e. mannuronate or mannuronic acid). In other words the alginate oligomer will contain at least or alternatively more than 50% mannuronate (or mannuronic acid) residues. Specific embodiments thus include alginate oligomers with (e.g. containing) at least 60, 70, 75, 80, 85, 90, 92 or 95% M (mannuronate) residues. Thus, a representative alginate oligomer for use according to this embodiment of the present invention will contain more than 70% M residues (i.e. more than 70% of the monomer residues of the alginate oligomer will be M residues). In one embodiment the alginate oligomer may be an oligomannuronate (i.e. a homooligomer of M, or 100% M).

In a further embodiment, the above described alginates of the invention have a primary structure wherein the majority of the M residues are in so called M-blocks. In this embodiment preferably at least 50%, more preferably at least 70 or 75%, and most preferably at least 80, 85, 90 or 95% of the M residues are in M-blocks. An M block is a contiguous sequence of at least two M residues, preferably at least 3 contiguous M residues, more preferably at least 4 or 5 contiguous M residues, most preferably at least 7 contiguous M residues.

In particular, at least 90% of the M residues are linked 1-4 to another M residue. More particularly at least 95%, more preferably at least 98%, and most preferably at least 99% of the M residues of the alginate are linked 1-4 to another M residue.

Other preferred oligomers are alginate oligomers wherein at least 70% of the monomer residues in the oligomer are M residues linked 1-4 to another M-residue, or more preferably at least 75%, and most preferably at least 80, 85, 90, 92, 93, 94, 95, 96, 97, 98, 99% of the monomers residues of the oligomer are M residues linked 1-4 to another M residue. This 1-4 linkage of two M residues can be alternatively expressed as a mannuronic unit bound to an adjacent mannuronic unit.

In a still further embodiment, the alginate oligomers of the invention comprise a sequence of alternating M and G residues. A sequence of at least three, preferably at least four, alternating M and G residues represents an MG block. The alginate oligomers of the invention may comprise an MG block. Expressed more specifically, an MG block is a sequence of at least three contiguous residues consisting of G and M residues and wherein each non-terminal (internal) G residue in the contiguous sequence is linked 1-4 and 4-1 to an M residue and each non-terminal (internal) M residue in the contiguous sequence is linked 1-4 and 4-1 to a G residue. Preferably the MG block is at least 5 or 6 contiguous residues, more preferably at least 7 or 8 contiguous residues.

In a further embodiment the minority uronate in the alginate oligomer (i.e. mannuronate or guluronate) is found predominantly in MG blocks. In this embodiment preferably at least 50%, more preferably at least 70 or 75% and most preferably at least 80, 85, 90 or 95% of the minority uronate monomers in the MG block alginate oligomer are present in MG blocks. In another embodiment the alginate oligomer is arranged such that at least 50%, at least 60%, at least 70%, at least 80%, at least 85%, at least 90%, at least 95%, at least 99%, e.g. 100% of the G and M residues in the oligomer are arranged in MG blocks.

In certain embodiments the terminal uronic acid residues of the oligomers of the invention do not have a double bond, especially a double bond situated between the C₄ and C₅ atom. Such oligomers may be described as having saturated terminal uronic acid residues. The skilled man would be able to prepare oligomers with saturated terminal uronic acid residues without undue burden. This may be through the use of production techniques which yield such oligomers, or by converting (saturating) oligomers produced by processes that yield oligomers with unsaturated terminal uronic acid residues.

The oligouronate will typically carry a charge and so counter ions for the oligouronate may be any physiologically tolerable ion, especially those commonly used for charged drug substances, e.g. sodium, potassium, ammonium, chloride, mesylate, meglumine, etc. Ions which promote alginate gelation e.g. group 2 metal ions may also be used.

While the oligouronate may be a synthetic material generated from the polymerisation of appropriate numbers of uronic acid units (e.g. guluronate and/or mannuronate residues), as noted above it is preferred to obtain the oligouronate from natural sources such as those mentioned above, e.g. natural alginate source materials.

As noted above, oligomers may be separated from polysaccharide breakdown products chromatographically using an ion exchange resin or by fractionated precipitation or solubilisation or filtration. U.S. Pat. No. 6,121,441 and WO 2008/125828, which are explicitly incorporated by reference herein in their entirety, describes a process suitable for preparing the alginate oligomers of use in the invention. Further information and discussion can be found in for example in “Handbooks of Hydrocolloids” (supra), which textbook is explicitly incorporated by reference herein in its entirety.

The oligouronates may also be chemically modified, including but not limited to modification to add charged groups (such as carboxylated or carboxymethylated glycans) or to alter flexibility (e.g. by periodate oxidation).

The alginates for production of alginate oligomers of the invention can also be obtained directly from suitable bacterial sources e.g. Pseudomonas aeruginosa or Azotobacter vinelandii, although algal sources are expected to be most suitable on account of the fact that the alginates produced in these organisms tend to have primary structures in which the majority of the G residues are arranged in G-blocks rather than as single residues.

The molecular apparatus involved in alginate biosynthesis in Pseudomonas fluorescens and Azotobacter vinelandii has been cloned and characterised (WO 94/09124; Ertesvåg, H., et al, Metabolic Engineering, 1999, Vol 1, 262-269; WO 2004/011628; Gimmestad, M., et al (supra); Remminghorst and Rehm, Biotechnology Letters, 2006, Vol 28, 1701-1712; Gimmestad, M. et al, Journal of Bacteriology, 2006, Vol 188(15), 5551-5560) and alginates of tailored primary structures can be readily obtained by manipulating these systems. The G content of alginates (for example an alginate source material) can be increased by epimerisation, for example with mannuran C-5 epimerases from A. vinelandii or other epimerase enzymes.

Thus, for example in vitro epimerisation may be carried out with isolated epimerases from Pseudomonas or Azotobacter, e.g. AIgG from Pseudomonas fluorescens or Azotobacter vinelandii or the AIgE enzymes (AIgE1 to AIgE7) from Azotobacter vinelandii. The use of epimerases from other organisms that have the capability of producing alginate, particularly algae, is also specifically contemplated. The in vitro epimerisation of low G alginates with Azotobacter vinelandii AIgE epimerases is described in detail in Ertesvåg et al (supra) and Strugala et al (Gums and Stabilisers for the Food Industry, 2004, 12, The Royal Society of Chemistry, 84-94). Epimerisation with one or more Azotobacter vinelandii AIgE epimerases other than AIgE₄ is preferred as these enzymes are capable of producing G block structures. Mutated versions or homologues from other organisms are also specifically contemplated as of use. WO 94/09124 describes recombinant or modified mannuronan C-5 epimerase enzymes (AIgE enzymes) for example encoded by epimerase sequences in which the DNA sequences encoding the different domains or modules of the epimerases have been shuffled or deleted and recombined. Alternatively, mutants of naturally occurring epimerase enzymes, (AIgG or AIgE) may be used, obtained for example by site directed or random mutagenesis of the AIgG or AIgE genes.

A different approach is to create Pseudomonas and Azotobacter organisms that are mutated in some or all of their epimerase genes in such a way that those mutants produce alginates of the required structure of alginate oligomer production, or even alginate oligomers of the required structure and size (or molecular weight). The generation of a number of Pseudomonas fluorescens organisms with mutated AIgG genes is described in detail in WO 2004/011628 and Gimmestad, M., et al, 2003 (supra). The generation of a number of Azotobacter vinelandii organisms with mutated AIgE genes is disclosed in Gimmestad, M., et al, 2006 (supra). The skilled man would be able to use this teaching to produce new mutants that would produce alginate oligomers of the invention without undue burden.

A further approach is to delete or inactivate the endogenous epimerase genes from an Azotobacter or a Pseudomonas organism and then to introduce one or more exogenous epimerase genes, which may or may not be mutated (i.e. may be wild-type or modified) and the expression of which may be controlled, for example by the use of inducible or other “controllable promoters”. By selecting appropriate combinations of genes, alginates of predetermined primary structure can be produced.

A still further approach would be to introduce some or all of the alginate biosynthesis machinery of Pseudomonas and/or Azotobacter into a non-alginate producing organism (e.g. E. coli) and to induce the production of alginate from these genetically modified organisms.

When these culture-based systems are used, the primary structure of the alginate or alginate oligomer can be influenced by the culture conditions. It is well within the capabilities of the skilled man to adjust culture parameters such as temperature, osmolarity, nutrient levels/sources and atmospheric parameters in order to manipulate the primary structure of the alginates produced by a particular organism.

References to “G residues/G” and “M residues/M” or to guluronic acid or mannuronic acid, or guluronate or mannuronate are to be read interchangeably as references to guluronic acid/guluronate and mannuronic acid/mannuronate (specifically α-L-guluronic acid/guluronate and β-D-mannuronic acid/mannuronate), and further include derivatives thereof in which one or more available side chains or groups have been modified without resulting in activity (anticancer or anti-ECM activity) that is substantially lower than that of the unmodified oligomer. Common saccharide modifying groups would include acetyl, sulphate, amino, deoxy, alcohol, aldehyde, ketone, ester and anhydro groups. The alginate oligomers may also be chemically modified to add charged groups (such as carboxylated or carboxymethylated glycans), and to alter flexibility (e.g. by periodate oxidation). The skilled man would be aware of still further chemical modifications that can be made to the monosaccharide subunits of oligosaccharides

As used herein ‘treatment’ refers to reducing, alleviating, ameliorating or eliminating the cancer, or one or more symptoms thereof, which is being treated, relative to the cancer or symptom prior to the treatment. Treatment may include a reduction or elimination of cancer cells, for example in solid tumours.

“Prevention” refers to delaying or preventing the onset of the symptoms of the cancer.

As referred to herein a subject may be any human or non-human animal, preferably a mammalian animal, e.g. a cow, horse, sheep, pig, goat, rabbit, cat, dog, especially preferably a human.

In particular embodiments the invention may involve first identifying or determining that the subject to be treated has cancer or is susceptible to or at risk of developing cancer.

Alternatively or additionally, the invention may involve assessing or monitoring the effect of the administration of the oligouronate and/or other anticancer agent on the subject, or more particularly on the cancer, or on the development or progress of the cancer. Procedures and means for assessing and/or monitoring an anticancer effect are well known in the art, for example by determining or monitoring symptoms, clinical condition, tumour size or spread (e.g. by imaging techniques) or other cancer or tumour indicators e.g. cancer/tumour markers etc.

The use of an oligouronate in combination with one or more other anticancer agents can result in improved drug delivery through the ECM, and the oligouronate can be used, for example, as an adjuvant or drug delivery agent in cancer therapy. Advantageously, lower doses or side effects of the drugs may be achieved when they are delivered or administered in combination with an oligouronate. The required dosage may be reduced by 10, 20, 30, 40 or 50%.

The anticancer drug or agent may be any suitable anti-cancer agent known in the art. A wide range of different types of agents are known or proposed for use in the treatment of cancer and any of these may be used, regardless of chemical nature or mode of action. Anticancer agents thus included chemical molecules whether naturally or synthetically derived or prepared (e.g organic small chemical molecules) and biological molecules such as proteins and peptides (e.g. immunotherapy agents as discussed below). Anticancer drugs thus include chemotherapeutic agents or drugs, which may be in a wide range of different chemical or functional classes, as well as antibodies or antibody derivatives and other biological molecules which act for example to stimulate, activate or enhance various physiological processes or cells in the body, for example immune and/or anti-inflammatory responses or cells etc.

Induction of an immune response to treat cancer is known as cancer “immunotherapy”. Immunotherapy can involve, for example, cell-based therapies, antibody therapies or cytokine therapies. All three approaches exploit the fact that cancer cells often have different cell-surface markers, or cancer antigens, which are detectable by the immune system. These antigens are most commonly proteins but may also include other molecules such as carbohydrates. Another example of immunotherapy is by checkpoint inhibition, whereby checkpoint proteins are inhibited. This is discussed further below.

Immunotherapy is thus used to provoke the immune system into attacking cancer cells, and as discussed further below, various molecules may be the target of immunotherapy-based approaches. For example, targets for immunotherapeutic intervention in cancer include “CD” (“cluster of differentiation”) proteins such as CD52, CD30, CD33, CD20, CD152 (also known as CTLA4) and CD279 (also known as programmed cell death 1 protein PD-1); growth factors such as vascular endothelial growth factor (VEGF); growth factor receptors such as epidermal growth factor receptor (EGFR) or human epidermal growth factor receptor 2 (HER2); Lymphocyte-activation gene 3 (LAGS); and B7 family proteins such as B7-H3 and B7-H4. These are merely representative examples however, and other molecules may be targets for immunotherapeutic intervention in cancer.

Thus, in one embodiment of the invention the anticancer agent is an immunotherapy agent, as discussed further below.

The present invention has utility with a wide range of anticancer agents, and examples of possible agents are discussed below.

Representative examples of anticancer agents in the “chemotherapy” class include but are not limited to fludarabine, gemcitabine, capecitabine, methotrexate, taxol, taxotere, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbazine, etoposide, teniposide, campathecins, bleomycin, doxorubicin, idarubicin, daunorubicin, dactinomycin, plicamycin, mitoxantrone, L-asparaginase, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, or topoteca.

Anticancer agents may include kinase inhibitors, phosphatase inhibitors, ATPase inhibitors, tyrphostins, protease inhibitors, herbimycin A, genistein, erbstatin, and lavendustin A.

In one embodiment, the anticancer agent may be selected from, but is not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or agents for use in photodynamic therapies, angiogenesis inhibitors, antimitotic agents, isoprenylation inhibitors, cell cycle inhibitors, actinomycins, bleomycins, anthracyclines, MDR inhibitors and Ca2+ATPase inhibitors.

Further anticancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Alternative anticancer agents may be selected from, but are not limited to, growth or hematopoietic factors such as erythropoietin and thrombopoietin, and growth factor mimetics thereof.

In one representative embodiment the drug is a small molecule, and more particularly a small molecule chemotherapeutic agent. A small molecule agent may be defined as having a molecular weight of less than 2000 Da, more particularly less than 1800, 1500, 1200, 1000, 900, 800 or 700 Da., typically less than 1000 Da. For example, a small molecule agent may have a size in the range of 100-1000 Da, e.g.100-800 Da or 300-700 Da.

In one embodiment of the invention the anticancer agent is accordingly not a macromolecular drug. In a further representative embodiment the anticancer agent is not a polymeric or oligomeric molecule e.g. a not a peptide, polypeptide or protein or not a nucleic acid or not a poly- or oligosaccharide.

However, in a preferred embodiment the anticancer agent is a polymeric or oligomeric molecule e.g. a peptide, polypeptide or protein or a nucleic acid or a poly- or oligosaccharide. Preferably the anticancer agent is a peptide, polypeptide or protein, including protein or peptide fusions or proteins or peptides conjugated to other molecules

In a further preferred embodiment the anticancer agent is an immunotherapy agent, which can be a peptide, polypeptide or protein. The immunotherapy agent may be selected from an antibody, a cytokine and a checkpoint inhibitor. As noted above a therapeutic anticancer antibody may have a range of targets, including checkpoint proteins. Thus, an antibody may be a checkpoint inhibitor.

Thus, in a first example, the immunotherapeutic agent is an antibody. As discussed above, cancer cells exhibit cancer antigens on their surface, which are recognised by antibodies that can thus be used therapeutically to target an immune response to the cancer cells or cells, e.g. wherein the cancer cells are in the form of a solid tumour. The antibody may be selected from monoclonal or polyclonal antibodies, mono-specific, bi-specific or multi-specific antibodies, monobodies and polybodies or indeed from any of the many antibody-like or antibody derivative molecules known in the art today. Accordingly the term “antibody” is used broadly herein and includes any such antibody and any antibody fragment, derivative or variant as in the known in the art. The antibody may be of any convenient or desired species, class or sub-type. Furthermore, the antibody may be natural, derivatised or synthetic.

The antibody may accordingly be:

(a) any of the various classes or subclasses of immunoglobulin e.g. IgG, IgA, IgM, IgD or IgE derived from any animal e.g. any of the animals conventionally used e.g. sheep, rabbits, goats, or mice or egg yolk;

(b) monoclonal or polyclonal antibodies;

(c) intact antibodies or fragments of antibodies, monoclonal or polyclonal, the fragments being those which contain the binding region of the antibody e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains;

(d) antibodies produced or modified by recombinant DNA or other synthetic techniques, including monoclonal antibodies, fragments of antibodies, humanised antibodies, chimeric antibodies, or synthetically made or altered antibody-like structures.

Also included are functional derivatives or “equivalents” of antibodies e.g. single chain antibodies. A single chain antibody may be defined as a genetically engineered molecule containing the variable region of the light chain, the variable region of the heavy chain, linked by a suitable polypeptide linker as a fused single chain molecule. Methods for producing antibodies and the fragments and derivatives of the antibodies are well known in the art.

In a preferred embodiment the antibody is a monoclonal antibody.

In many cases monoclonal antibodies are antibodies without modification, and most of the currently-used therapeutic antibodies fall into this category. However, in one embodiment of the present invention the antibody, for example a monoclonal antibody, is conjugated or fused to a further additional molecule, for example a toxic substance or a radioactive substance. Thus, conjugated or fused antibodies are joined to another molecule, which is either toxic to cells (e.g. a drug) or radioactive. The antibody binds to specific antigens on the surface of cancer cells and directs the toxin or radiation to the tumour.

The toxic molecule may be, for example, a routinely used drug, such as a chemotherapy drug as discussed herein (e.g. monomethyl auristatin E as mentioned below), but any other suitable molecules can be used, for example macromolecular drugs such as proteins or peptides or other biological molecules may be used. If the antibodies are labelled with chemotherapy or toxins, they are known as chemolabelled or immunotoxins, respectively.

Radioactive compound-linked antibodies are referred to as radiolabelled. Examples of suitable radioisotopes include yttrium-90, iodine-131, strontium-89, samarium-153 and radium-223.

Thus, in one embodiment of the invention the antibody is a conjugated antibody or fusion antibody (or fusion protein), i.e. conjugated or fused to a further additional molecule such as a toxin (e.g. a protein or peptide, or drug, for example a chemotherapeutic drug) or a radioisotope.

In an alternative embodiment the antibody is conjugated or fused to a small interfering RNA (siRNA). siRNA is used for targeted gene silencing. However, one of the hurdles to making siRNA a human therapeutic includes effective in vivo delivery and being able to deliver drugs to target cells only. Antibody-based delivery agents can be used to target and deliver siRNA into specific cell types, and hence in the present invention the antibodies as discussed herein may be conjugated or fused to an siRNA molecule. In an alternative embodiment the siRNA may not be conjugated to an antibody, i.e. it may be in free (unbound) form.

Any suitable antibody which recognises and binds to a cancer antigen may be used in the invention. As mentioned above, examples of cancer antigens, or targets for therapeutic antibodies, include many “CD” proteins, such as CD52, CD47, CD30, CD33, CD20, CD152 and CD279; growth factors such as vascular endothelial growth factor (VEGF); growth factor receptors such as epidermal growth factor receptor (EGFR) or human epidermal growth factor receptor 2 (HER2).

Several antibodies that bind to such antigens or targets are known and have been approved for the treatment of cancer, and any of these antibodies may be used in the present invention. Preferred antibodies are those that have utility in treating solid tumours, and especially those with an altered ECM, such as breast, ovarian and pancreatic cancers.

Known and approved antibodies include: Alemtuzumab, Bevacizumab, Brentuximab vedotin, Cetuximab, Gemtuzumab ozogamicin, Ibritumomab tiuxetan, Ipilimumab, Ofatumumab, Panitumumab, Rituximab, Tositumomab and Trastuzumab.

Alemtuzumab is an anti-CD52 humanized IgG1 monoclonal antibody indicated for the treatment of fludarabine-refractory chronic lymphocytic leukemia (CLL), cutaneous T-cell lymphoma, peripheral T-cell lymphoma and T-cell prolymphocytic leukemia.

Bevacizumab (Avastin) is a humanized IgG1 monoclonal antibody which binds to vascular endothelial growth factor-A (VEGF-A) (referred to commonly as VEGF without a suffix). Bevacizumab binds to and physically blocks VEGF, preventing receptor activation which has consequences for tumour vascularisation. Bevacizumab is licensed for colon cancer, kidney cancer, lung cancer, ovarian cancer, glioblastoma and breast cancer.

Brentuximab vedotin is a second generation chimeric IgG1 antibody drug conjugate used in the treatment of Hodgkin lymphoma and anaplastic large cell lymphoma (ALCL). It is an antibody conjugated to monomethyl auristatin E, a drug that prevents cell division by disrupting microtubules. The antibody binds to CD30, often found highly expressed on the surface of Hodgkin lymphoma and ALCL cells, and is then internalised where the drug is detached from the antibody and exerts its cellular effects. By preventing cell division it kills cancer cells by the induction of programmed cell death.

Cetuximab (Erbitux) is a chimeric IgG1 monoclonal antibody that targets the extracellular domain (part of the receptor outside the cell) of the epidermal growth factor receptor (EGFR). It is used in the treatment of colorectal cancer and head and neck cancer. Once a ligand binds to the EGFR on the surface of the cell, signalling pathways are activated inside the cell that are associated with malignant characteristics. These include the PI3K/AKT and KRAS/BRAF/MEK/ERK pathways that cause cancer cell proliferation, invasion, differentiation and cancer stem cell renewal. Cetuximab functions by competitively inhibiting ligand binding, thereby preventing EGFR activation and subsequent cellular signalling.

Gemtuzumab ozogamicin is an “immuno-conjugate” of an IgG4 anti-CD33 antibody chemically linked to a cytotoxic calicheamicin derivative, and may be used for the treatment of acute myeloid leukaemia (AML).

Ibritumomab tiuxetan (Zevalin) is a murine anti-CD20 antibody chemically linked to a chelating agent that binds the radioisotope yttrium-90 (90Y). It is used to treat a specific type of non-Hodgkin lymphoma, follicular lymphoma, which is a tumour of B-cells.

Ipilimumab (Yervoy) is a human IgG1 antibody that binds the surface protein CTLA4 which has a role in negatively regulating the activation of T-cells. CTLA4 is discussed below in the context of checkpoint inhibitors.

Nimotuzumab is a chimeric human-mouse anti-EGFR monoclonal antibody and has been approved for squamous cell carcinoma in head and neck (SCCHN).

Ofatumumab is a second generation human IgG1 antibody that binds to CD20. It is used in the treatment of chronic lymphocytic leukemia (CLL) as the cancerous cells of CLL are usually CD20-expressing B-cells. Unlike Rituximab, which binds to a large loop of the CD20 protein, Ofatumumab binds to a separate small loop.

Panitumumab (Vectibix) is a human IgG2 antibody that binds to the EGF receptor. Like Cetuximab, it prevents cell signalling by the receptor by blocking the interaction between the receptor and its ligand. It is used in the treatment of colorectal cancer.

Rituximab is a chimeric monoclonal IgG1 antibody specific for CD20, developed from its parent antibody Ibritumomab. As with Ibritumomab, Rituximab targets CD20, which is present on B-cells. For this reason it is effective in treating certain types of malignancies that are formed from cancerous B-cells. These include aggressive and indolent lymphomas such as diffuse large B-cell lymphoma and follicular lymphoma, and leukaemias such as B-cell chronic lymphocytic leukaemia.

Tositumomab was a murine IgG2a anti-CD20 antibody covalently bound to radioactive Iodine 131 known as “Bexxar” that was approved for treatment of Non-Hodgkin lymphoma, but was voluntarily withdrawn from the market.

Trastuzumab (Herceptin) is a monoclonal IgG1 humanized antibody specific for the epidermal growth factor receptor 2 protein (HER2). It received FDA-approval in 1998, and is clinically used for the treatment of breast cancer. HER-2 is a member of the epidermal growth factor receptor (EGFR) family of transmembrane tyrosine kinases. In a preferred embodiment of the invention the immunotherapeutic agent, and hence anticancer agent, is trastuzumab, preferably for the treatment of ovarian cancer.

In an alternative embodiment the antibody is an anti-CD47 antibody, i.e. an antibody which blocks CD47 signalling. Such antibodies have been shown to eliminate or inhibit the growth of a wide range of cancers and tumours in laboratory tests on cells and mice. CD47 is present on many cancer cells and on many healthy cells.

In a further alternative embodiment the antibody is an antibody to a carbohydrate molecule found on the surface of cancer cells. By way of example, such an antibody may be an anti-GD2 antibody. GD2 is a ganglioside found on the surface of many types of cancer cell including neuroblastoma, retinoblastoma, melanoma, small cell lung cancer, brain tumours, osteosarcoma, rhabdomyosarcoma, Ewing's sarcoma, liposarcoma, fibrosarcoma, leiomyosarcoma and other soft tissue sarcomas. It is not usually expressed on the surface of normal tissues, making it a good target for immunotherapy to allow for specific action against the tumour and reduced toxicity.

In a second example, the immunotherapeutic agent is a cytokine. Cytokines include immunomodulating agents, such as interleukins (IL) and interferons (IFN) and also colony stimulating factors, tumour necrosis factors (TNF) and other regulatory molecules. Cytokines have been classed as lymphokines, interleukins, and chemokines, based on their function, cell of secretion, or target of action. Each cytokine has a matching cell-surface receptor, which initiates cascades of intracellular signalling which alter cell functions. In the context of cancer, cytokines are produced by many cell types present within a tumour. Cytokines are well known in the art and all such cytokines are encompassed for use according to the invention. As such, in one embodiment the immunotherapeutic agent is a cytokine. In a preferred embodiment the cytokine is an interleukin or an interferon.

Interleukins are a group of cytokines with a wide array of effects on the immune system. Examples of interleukins (ILs) are IL-1, IL-2, IL-3, IL-4, IL-5, IL-6, IL-7, IL-8, IL-9, IL-10, IL-11, IL-12, IL-13, IL-15 and IL-17. In a preferred embodiment the interleukin is IL-2.

Interleukin-2 has numerous effects on the immune system and acts as a general T-cell growth factor. It does this by binding to receptors on the surface of T cells. This binding stimulates the proliferation of T cells, continued cytokine production, and activation of multiple types of immune cells. IL-2 is used in the treatment of malignant melanoma and renal cell carcinoma. High-dose IL-2 therapy has been shown to lead to a complete response in a subset of patients (4%-6%) in renal cell carcinoma and melanoma. This suggests that for this subset of patients, IL-2 therapy is able to successfully manipulate the endogenous anti-tumour immune response. In normal physiology it promotes both effector T cells (cells that produce the immune response) and T-regulatory cells (cells that repress the immune response), but its exact mechanism in the treatment of cancer is unknown.

Interferons are cytokines produced by the immune system usually involved in anti-viral response, but also have use in the treatment of cancer. There are three groups of interferons (IFNs): type I (IFNα and IFNβ), type 2 (IFNγ) and the relatively newly discovered type III (IFNλ). In a preferred embodiment the immunotherapeutic agent is interferon-α. IFNα has been approved for use in hairy-cell leukaemia, AIDS-related Kaposi's sarcoma, follicular lymphoma, chronic myeloid leukaemia and melanoma.

All known forms of the above-discussed cytokines can be used in the present invention, including also functionally equivalent variants, derivatives and fragments thereof. Thus the term “cytokine” as used herein includes amino acid sequence variants of known cytokine polypeptides, and fragments of a cytokine polypeptide, or derivative thereof, as long as such fragments, variants or derivatives are active, or “functional”, i.e. retain at least one function or activity (e.g. biological activity) of the relevant cytokine. The cytokine may be a recombinant polypeptide, a synthetic polypeptide or may be isolated from a natural source. Suitable cytokines are commercially available and would be known to the skilled man, for example human cytokines are available from GenScript (Piscataway, N.J., USA).

In a third example, the immunotherapeutic agent is an agent that targets an immune checkpoint, i.e. is a checkpoint inhibitor. Checkpoint proteins keep the immune system in check by indicating to the immune system which cells are healthy and which cells should be destroyed. Checkpoint proteins act as a “brake” on the immune system by preventing T-cell activation. If a cell does not have sufficient checkpoint proteins on its surface it may be destroyed by the immune system. In the case of cancer cells, whilst there may be molecules signalling that the cell is cancerous, if there are enough checkpoint proteins on the cell surface, the cell may evade the immune response, and it has been speculated that checkpoint proteins contribute to a lack of success in some cancer immunotherapies.

The best known example of a checkpoint protein is PD-L1 (for Programmed Death Ligand 1). The receptor for PD-L1 is PD-1. PD-L1 prevents T-cells from attacking healthy cells. Cancer cells may upregulate PD-L1 as a protective mechanism. When PD-L1 activates the PD-1 receptor on the surface of a T-cell, the T-cell is signalled to destroy itself. If the T-cells are programmed to selectively attack cancer cells, that set of T-cells will be destroyed and the cancer prevails.

Another checkpoint protein is cytotoxic T-lymphocyte antigen-4, or CTLA4. Once a cytotoxic T cell becomes active it expresses CTLA4 on its surface, which then competes with the co-stimulatory molecule CD28 for their mutually shared ligands, B7-1 and B7-2 on antigen-presenting cells. This balance holds cytotoxic activity in check, while allowing T cell function to proceed in a self-limited manner.

Other checkpoint proteins include CD-137 (4-1 BB) which is a costimulatory checkpoint protein; lymphocyte activation gene 3 (LAG-3, CD223), a CD4-related inhibitory receptor coexpressed with PD-1 on tolerant T cells; B7 superfamily proteins B7-H3 and B7-H4; T cell protein TIM3; and phosphatidylserine (PS) which is a phospholipid in normal cells that is translocated to the outer member surface during apoptosis, suppressing the excess immune activation that would otherwise occur during processing and clearance of decaying cell matter. Externalization of PS indirectly stimulates macrophages, resulting in suppression of dendritic ell antigen presentation. Like PD-L1, externalized PS is aberrantly expressed by some tumour cells and tumour-derived microvesicles. Thus, PS is believed to be exploited by tumours to prevent adaptive tumour immunity.

The immunotherapeutic agent according to the present invention may target or inhibit any of these checkpoint proteins. Such immunotherapeutic agents are known as “checkpoint inhibitors”.

Checkpoint inhibitors (also known as immune checkpoint modulators, or CPMs) are designed to lessen the effectiveness of checkpoint proteins. Ideally a CPM should expose cancers to the immune system without causing that same system to attack healthy tissue.

Several checkpoint inhibitors are known and can be used in the present invention, for example those inhibitors described in Creelan (2014) Cancer Control 21:80-89, which is hereby incorporated by reference.

Examples of checkpoint inhibitors include: Tremelimumab (CP-675,206), a human IgG2 monoclonal antibody with high affinity to CTLA-4; Ipilimumab (MDX-010), a human IgG1 monoclonal antibody to CTLA-4; Nivolumab (BMS-936558), a human monoclonal anti-PD1 IgG4 antibody that essentially lacks detectable antibody-dependent cellular cytotoxicity (ADCC); MK-3475 (formerly lambrolizumab), a humanized IgG4 anti-PD-1 antibody that contains a mutation at C228P designed to prevent Fc-mediated ADCC; Urelumab (BMS-663513), a fully human IgG4 monoclonal anti-CD137 antibody; anti-LAG-3 monoclonal antibody (BMS-986016); and Bavituximab (chimeric 3G4), a chimeric IgG3 antibody against PS. All of these checkpoint inhibitors can be used in the present invention.

An alternative strategy is to inhibit PD-L1, the ligand for PD-1, on the tumour cell surface, and therefore inhibitors of PD-L1 are also encompassed by the present invention. For example, MPDL3280A (RG7446), is a human IgG1-kappa anti-PD-L1 monoclonal antibody and may be used according to the invention. MED14736 is another IgG1-kappa PD-L1 inhibitor.

Another alternative approach is to competitively block the PD-1 receptor, using a B7-DC-Fc fusion protein, and such fusion proteins can also therefore be used in the present invention. In a further alternative approach an antibody to Killer cell immunoglobulin-like receptor may be used as the immunotherapeutic agent. Killer cell immunoglobulin-like receptor (KIR) is a receptor on NK cells that downregulates NK cytotoxic activity. HLA class I allele-specific KIR receptors are expressed in cytolytic (CD56dimCD16+) NK cells, while CD56brightCD16-NK subset lacks these KIRs. Along these lines, inhibitory KIRs seem to be selectively expressed in the peritumoral NK cell infiltrate and thus seem to be a checkpoint pathway co-opted by tumours, similar to PD-L1. As such, inhibition of specific KIRs using antibodies should cause sustained in vivo activation of NK cells. For example, Iirilumab (IPH2102) is fully human monoclonal antibody to KIR and can be used according to the invention.

An alternative option for immunotherapy relates to immune cell therapies, and the present invention can also be used in combination with such therapies, for example adoptive cell transfer. A number of T-cell-based therapies for treating cancer have been developed, and these treatments, known as adoptive cell transfer (ACT) have become increasingly attractive during recent years. Three main ACT strategies have been exploited thus far. The first of these, and the most developed, involves the isolation of patient's own tumour-reactive T-cells from peripheral or tumour sites (known as Tumour Infiltrating Lymphocytes (TILs)). These cells are expanded ex vivo and re-injected into a patient.

Two alternative therapies are available, which involve modification of a patient's own T-cells with receptors capable of recognising a tumour. In one option, TcRs having activity towards a cancer antigen can be isolated and characterised, and a gene encoding the TcR can be inserted into T-cells and re-injected into a patient. This therapy has been shown to shrink solid tumours in some patients, but is associated with a significant drawback: the TcRs used must be matched to a patient's immune type. Accordingly, as an alternative to the use of TcRs, therapies involving the expression of Chimeric Antigen Receptors (CARs) in T-cells have also been suggested. CARs are fusion proteins comprising an antibody linked to the signalling domain of the TcR complex, and can be used to direct T cells against a tumour if a suitable antibody is selected. Unlike a TCR, a CAR does not need to MHC-matched to the recipient.

Alternatively, the cell may be a natural killer (NK) cell, which optionally may be modified to express a CAR.

As such, according to the present invention the immunotherapeutic agent may be a cell, particularly an immune cell such as a lymphocyte, particularly a T cell or NK cell as described above, e.g. the T cell may be a TIL or be modified to express a TcR or CAR. The NK may be modified to express a CAR.

An alternative option for the anticancer agent is a microRNA (miRNA). MicroRNAs are small non-coding RNA molecule (containing about 22 nucleotides) found in plants, animals, and some viruses, which functions in RNA silencing and post-transcriptional regulation of gene expression. miRNAs function via base-pairing with complementary sequences within mRNA molecules. As a result, these mRNA molecules are silenced by cleavage of the mRNA strand into two pieces, destabilization of the mRNA through shortening of its poly(A) tail, or less efficient translation of the mRNA into proteins. miRNAs resemble the siRNAs mentioned above, except miRNAs derive from regions of RNA transcripts that fold back on themselves to form short hairpins, whereas siRNAs derive from longer regions of double-stranded RNA. Many miRNAs have been found to have links with various types of cancer and accordingly are sometimes referred to as “oncomirs”.

MicroRNAs can be used in microRNA-based oncology therapeutics in the treatment of cancer. The rationale for developing miRNA therapeutics is based on the premise that aberrantly expressed miRNAs play key roles in the development of cancers, and that correcting these miRNA deficiencies by either antagonizing or restoring miRNA function may provide a therapeutic benefit, e.g. by miRNA replacement therapy. For example, MRX34 (MiRNA Therapeutics) is the first microRNA replacement therapy that has advanced into clinical testing in cancer patients. MRX34 was designed to deliver a mimic of the naturally occurring microRNA tumour suppressor miR-34, which is under-expressed in a wide variety of cancers. MRX34 can be used in the present invention.

By way of further example, microRNA-7 is a 23 nucleotide miRNA whose expression is tightly regulated and restricted predominantly to the brain, spleen and pancreas. Reduced levels of miR-7 have been linked to the development of cancer and metastasis. As a tumour suppressor, miR-7 functions to co-ordinately downregulate a number of direct (e.g. the epidermal growth factor receptor) and indirect (e.g. phospho-Akt) growth promoting targets to decrease tumour growth in vitro and in vivo. In addition, miR-7 can increase the sensitivity of treatment-resistant cancer cells to therapeutics and inhibit metastasis. Thus, replacement of miR-7 (‘miRNA replacement therapy’) for specific human cancers could represent a new treatment approach.

Any suitable miRNA may be used as the anticancer agent according to the present invention. The miRNA may be in free form, i.e. not bound to another molecule. Alternatively, the miRNA may be conjugated or bound to another molecule, e.g. an antibody as discussed herein.

The present invention is applicable to any cancer. Cancer is defined broadly herein to include any neoplastic condition, and includes particularly malignant or pre-malignant conditions. The cancer may cause or result in or manifest in solid tumours, but is not limited to such, and includes also cancers of the haempoietic system. Benign tumours are also included.

The cancer may occur in any tissue or organ of the body. For example, the present invention can be used in the treatment or prevention of any of the following cancers in a patient or subject:

Acute Lymphoblastic Leukaemia (ALL), Acute Myeloid Leukaemia (AML), Adrenocortical Carcinoma, AIDS-Related Cancer (e.g. Kaposi Sarcoma and Lymphoma), Anal Cancer, Appendix Cancer, Astrocytomas, Atypical Teratoid/Rhabdoid Tumour, Basal Cell Carcinoma, Bile Duct Cancer, Extrahepatic Bladder Cancer, Bone Cancer (e.g. Ewing Sarcoma, Osteosarcoma and Malignant Fibrous Histiocytoma), Brain Stem Glioma, Brain Cancer, Breast Cancer, Bronchial Tumours, Burkitt Lymphoma, Carcinoid Tumour, Cardiac (Heart) Tumours, Cancer of the Central Nervous System (including Atypical Teratoid/Rhabdoid Tumour, Embryonal Tumours, Germ Cell Tumour, Lymphoma), Cervical Cancer, Chordoma, Chronic Lymphocytic Leukemia (CLL), Chronic Myelogenous Leukaemia (CML), Chronic Myeloproliferative Disorder, Colon Cancer, Colorectal Cancer, Craniopharyngioma, Cutaneous T-Cell Lymphoma, Bile Duct Cancer, Extrahepatic Ductal Carcinoma In Situ (DCIS), Embryonal Tumours, Endometrial Cancer, Ependymoma, Esophageal Cancer, Esthesioneuroblastoma, Ewing Sarcoma, Extracranial Germ Cell Tumour, Extragonadal Germ Cell Tumour, Extrahepatic Bile Duct Cancer, Eye Cancer (including Intraocular Melanoma and Retinoblastoma), Fibrous Histiocytoma of Bone, Gallbladder Cancer, Gastric (Stomach) Cancer, Gastrointestinal Carcinoid Tumour, Gastrointestinal Stromal Tumours (GIST), Germ Cell Tumor, Gestational Trophoblastic Disease, Glioma, Hairy Cell Leukaemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumours, Pancreatic Neuroendocrine Tumours, Kaposi Sarcoma, Kidney Cancer (including Renal Cell and Wilms Tumour), Langerhans Cell Histiocytosis, Laryngeal Cancer, Leukaemia (including Acute Lymphoblastic (ALL), Acute Myeloid (AML), Chronic Lymphocytic (CLL), Chronic Myelogenous (CML), Lip and Oral Cavity Cancer, Liver Cancer (Primary), Lobular Carcinoma In Situ (LCIS), Lung Cancer, Lymphoma, Macroglobulinemia, Waldenström, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Childhood, Multiple Myeloma/Plasma Cell Neoplasm, Mycosis Fungoides, Myelodysplastic Syndromes, Myelodysplastic/Myeloproliferative Neoplasms, Multiple Myeloma, Myeloproliferative Disorders, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumours (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Sézary Syndrome, Skin Cancer, Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Urethral Cancer, Uterine Cancer, Endometrial, Uterine Sarcoma, Vaginal Cancer, Vulvar Cancer, Waldenström Macroglobulinemia, and Wilms Tumour. p The present invention particularly encompasses treatment or prevention of cancers which involve an altered ECM, for example a stiffened or thickened ECM as discussed above, which may be seen in some breast, ovarian, and pancreatic cancers. Also mentioned in this regard may be desmoplastic (or highly desmoplastic) tumours or tumours with stromal involvement.

In a further embodiment the cancer presents as one or more solid tumour. In a preferred embodiment the cancer is selected from breast, ovarian and pancreatic cancer or any cancer with stromal involvement or with desmoplastic or highly desmoplastic tumours. Stromal involvement may comprise the formation of a dense fibrotic mass (termed the stroma) which forms around the cancer cells in the tumour and comprises connective tissue, fibroblasts, leucocytes and blood vessels. Essentially the stroma comprises blood and lymphatic vessels and a variety of non-malignant host cells all embedded in an ECM. In certain cancers, including notably pancreatic cancer and also ovarian cancer, it can be particularly compact, or dense, and extensive. Thus certain tumours can present a stromal barrier and the treatment of such stroma-rich tumours presents a particular aspect of the invention.

Alternatively viewed, certain cancers may be associated with a desmoplasia, which consists of fibroblasts, cells from the tissue in which the cancer has formed, lymphatic and vascular endothelial cells, immune cells, pathologic increased nerves and ECM, and creates complex tumour microenvironment promoting cancer development and which may pose a barrier to chemotherapy. Pancreatic cancer is a notable example, e.g. pancreatic ductal adenocarcinoma, and breast cancer.

The oligouronate and anticancer agent of may be used according to the present invention in the form of a composition, i.e. a pharmaceutical composition. As noted above, the oligouronate and other anticancer agent may be formulated together in a single composition or in separate compositions for separate administration. This will depend on the nature of the other anticancer agent and its selected or required mode of administration. It is a surprising feature of the present invention that the oligouronate may be separately administered to the subject (for example by systemic e.g. parenteral means of administration) and enhance the delivery of a separately administered anticancer agent, which may be administered by a different means of administration, In particular it is preferred to administer the oligouronate by intravenous (i.v) administration. The anticancer agent may be administered by other means, including other parenteral means, or may also be administered separately i.v.

The compositions for use in the invention may be formulated in any convenient manner according to techniques and procedures known in the pharmaceutical art, e.g. using one or more pharmaceutically acceptable diluents, carriers or excipients. “Pharmaceutically acceptable” as referred to herein refers to ingredients that are compatible with other ingredients of the compositions as well as physiologically acceptable to the recipient. The nature of the composition and carriers or excipient materials, dosages etc. may be selected in routine manner according to choice and the desired route of administration, purpose of treatment etc. Dosages may likewise be determined in a routine manner and may depend upon the nature of the molecule, purpose of treatment, age of patient, mode of administration etc. Any therapeutic agent of the invention as above described may be combined with pharmaceutically acceptable excipients to form therapeutic compositions.

Thus, “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the nature of the cancer to be treated, the severity of the illness, the age, weight, and sex of the patient, etc., or alternatively of the desired duration of treatment.

The pharmaceutical compositions contain vehicles which are pharmaceutically acceptable for formulation. These may be in particular isotonic, sterile, saline solutions (monosodium or disodium phosphate, sodium, potassium, calcium or magnesium chloride and the like or mixtures of such salts), or dry, freeze-dried compositions which upon addition, depending on the case, of sterilized water or physiological saline, permit the administration of solutions.

To prepare pharmaceutical compositions, an effective amount of an oligouronate or anticancer drug according to the invention may be dissolved or dispersed in a pharmaceutically acceptable carrier or aqueous medium. The compositions may comprise any known carrier, diluent or excipient. For example, formulations which are suitable for parenteral administration conveniently comprise sterile aqueous solutions and/or suspensions of pharmaceutically active ingredients preferably made isotonic with the blood of the recipient, generally using sodium chloride, glycerin, glucose, mannitol, sorbitol and the like.

Any mode of administration common or standard in the art may be used, e.g. injection, infusion, topical administration, inhalation, transdermal administration, both to internal and external body surfaces etc. by any suitable method known in the medicinal arts. Thus modes of administration include oral, nasal, enteral, rectal, vaginal, transmucosal, topical, or parenteral administration or by inhalation. Administration may be direct to the tumour.

For the oligouronate parenteral means of administration are preferred, e.g intravenous, intramuscular, intraperitoneal or subcutaneous administration. In a particular embodiment, the oligouronate and anticancer agent are administered parenterally, preferably intravenously. In an alternative embodiment the oligouronate is administered directly to a tumour, for example by injection or infusion.

In a particular embodiment of the invention the oligouronate is not complexed with or contained within cationic polymeric material and/or is not contained in or present on the surface of a vesicle or particle, particularly a vesicle or particle having a surface positive charge. Thus in one embodiment particulate or vesicular materials or preparations comprising an oligouronate or preparations in which the oligouronate is provided with a shell comprising a cationic polymeric material are excluded from the scope of the invention. More particularly the oligouronate is not provided or used in the form of a liposome, lipoplex or polyplex.

In an alternative embodiment of the invention the oligouronate is not complexed with or contained within, or in the form of, a microsphere or microparticle, i.e. a sphere or particle with a diameter in the micrometre range, for example from 1 to 1000 μm. In a particular embodiment the oligouronate is an alginate oligomer which is not complexed with or contained within, or in the form of, a microsphere or microparticle.

In a further alternative embodiment of the invention the oligouronate is not a water-insoluble and/or water-swellable polymer, thus the oligouronate is not in a “gelled” form that is water insoluble. Thus, in a preferred embodiment of the invention the oligouronate is water-soluble.

As noted above, the oligouronate and anticancer agent may be administered simultaneously, separately or sequentially. In a preferred embodiment the oligouronate and anticancer agent are administered sequentially, e.g. at separate times, i.e. not together in the same composition. In an alternate embodiment the oligouronate and anticancer agent administered together at the same time, for example in the same composition or in separate compositions. The timing of the separate administrations may be determined according to the particular anticancer agent, formulations and/or modes of administration used. Thus the oligouronate may be administered before or after the anticancer agent. For example the anticancer agent may be administered first parenterally (e.g i.v. or i.p.) and the oligouronate may be administered at a suitable time interval afterwards to align with the optimum time of anticancer agent delivery to the target site. Such determinations are entirely within the routine skill of the clinician. Thus for example the oligouronate may be administered, preferably parenterally, more preferably i.v. at least or up to 20, 30, 40, 50, 60, 70, or 90 minutes or 2, 3, 4, 5 or 6 hours before or after the anticancer agent.

The present invention also provides a product or kit comprising an oligouronate and a small molecule anticancer agent (that is a non-macromolecular drug), in particular a small molecule chemotherapeutic agent. The kit or product can be used in any of the uses or methods described herein, i.e. for use in treating or preventing cancer, and/or for enhancing the delivery of the anticancer agent. Particularly, the kit or product is for simultaneous, separate or sequential use. Preferably the oligouronate, and optionally also the anticancer agent, is formulated for parenteral administration, preferably i.v. administration.

The invention will be further described with reference to the following non-limiting Examples in which:

FIG. 1 shows the results of a study in which 300 μl of a control or test (with 100 μl G Block added) Matrigel solution were applied to rheometer plates at 10° C. (using C 40/1 geometry). A Temperature sweep was performed from 10-37° C. , ½° minute⁻¹.

FIG. 2 shows the results of a study in which a control or various test Matrigel solutions (with various added oligouronates) were applied to rheometer plates at 10° C. (using C 40/1 geometry). A Temperature sweep was performed from 10-37° C. , ½° minute⁻¹.

FIG. 3 shows the results of a study to test the effect of an oligouronate alone or in combination with an anticancer agent (Gemcitabine) on tumour size in an in vivo model of pancreatic cancer.

FIG. 4 shows the average weight of the animal in the same study as FIG. 3.

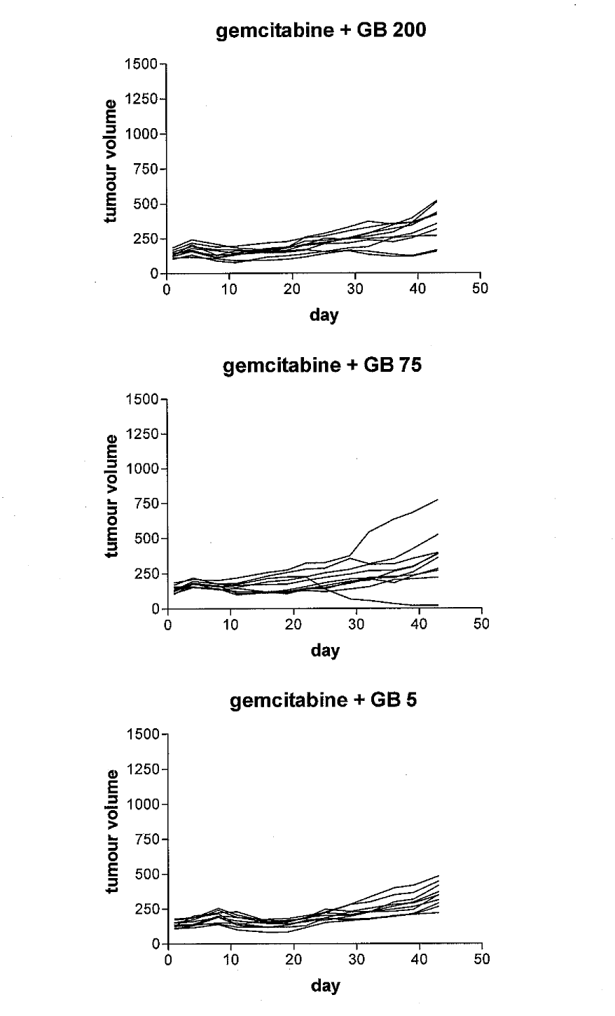

FIG. 5 shows individual curves of tumour weight versus day, panel by treatment.

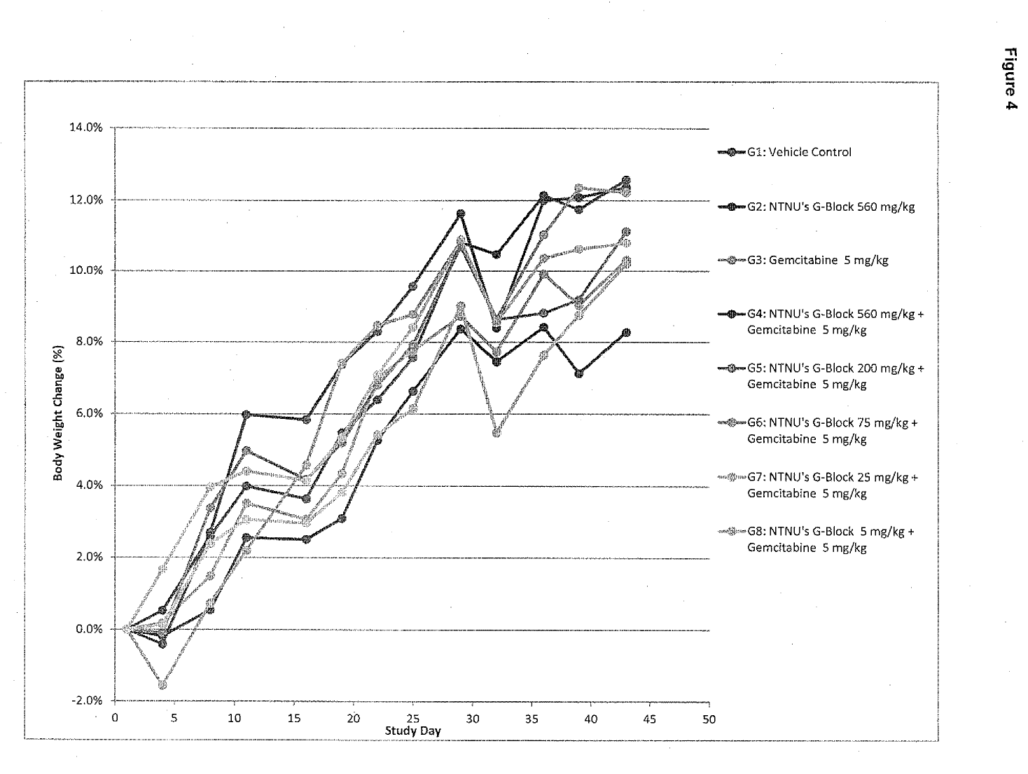

FIG. 6 shows mean value curves of tumour weight versus day.

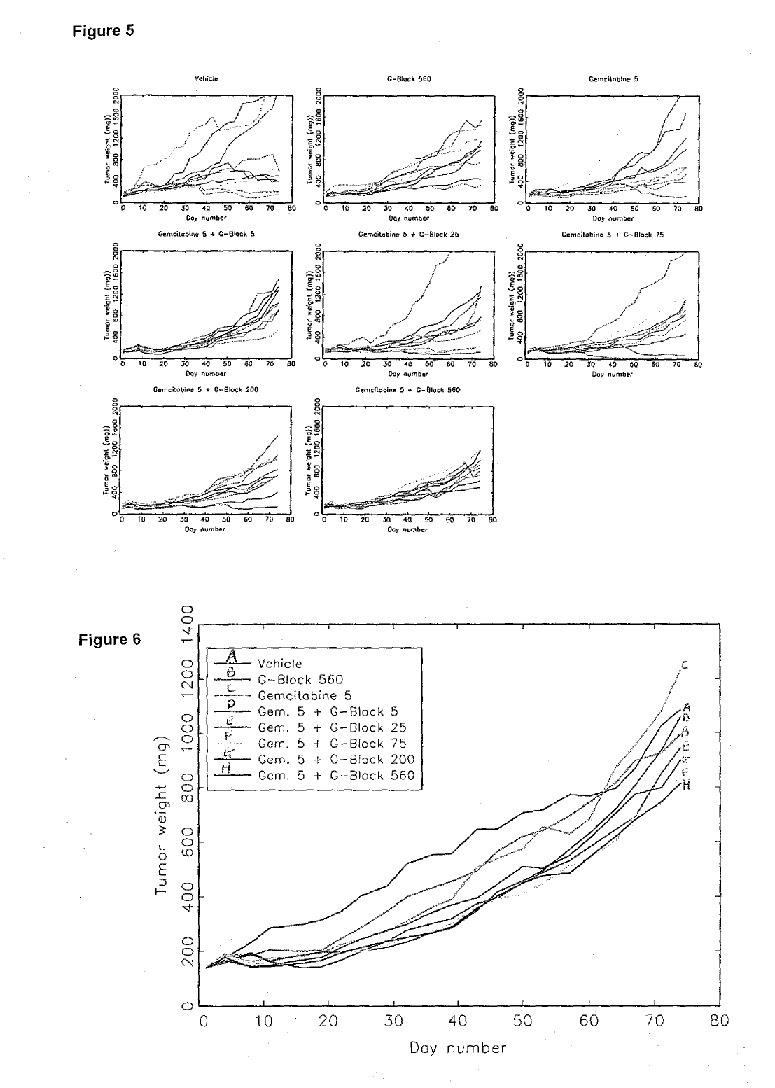

FIG. 7 shows the estimated differences with 95% confidence intervals for comparisons of active treatments with vehicle by day.

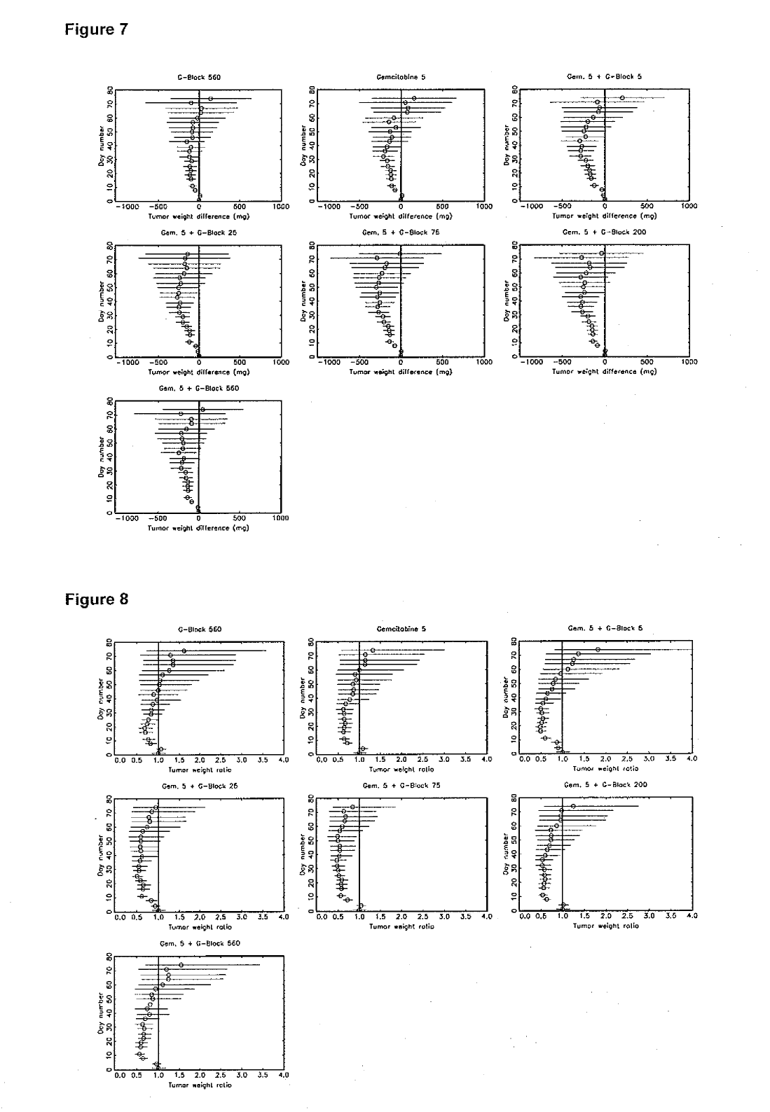

FIG. 8 shows the estimated ratios with 95% confidence intervals for comparisons of active treatments with vehicle by day.

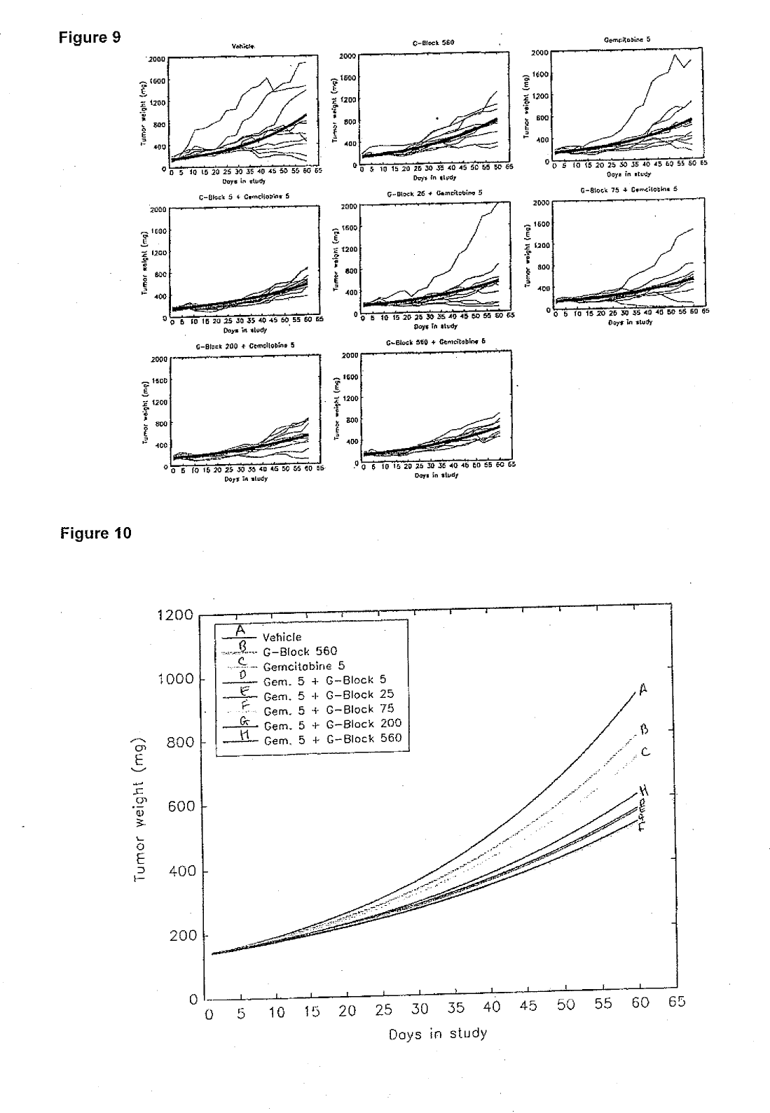

FIG. 9 shows individual tumour weight curves with mean value curves and fitted exponential curves superimposed.

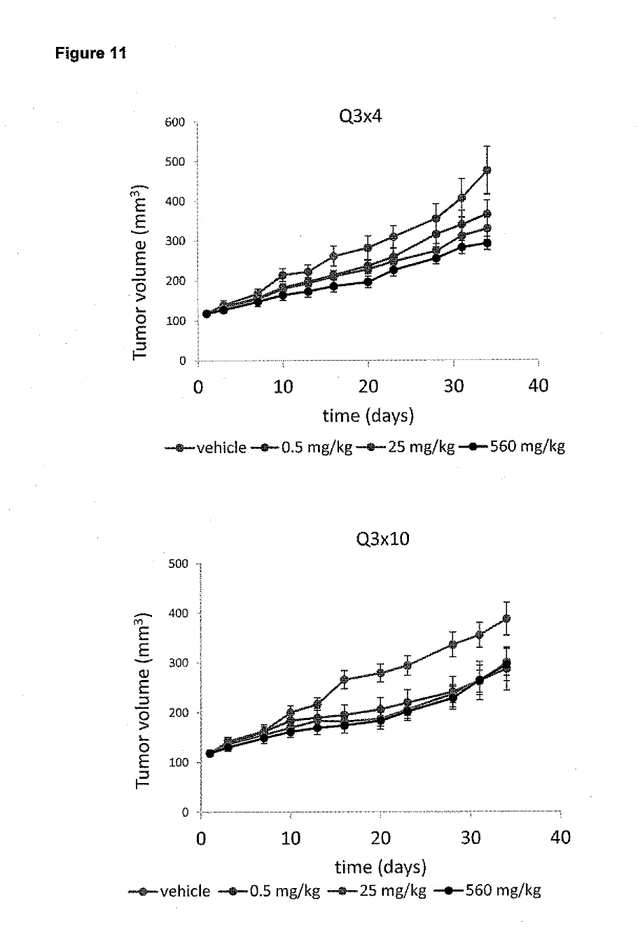

FIG. 10 shows fitted exponential curves by treatment.

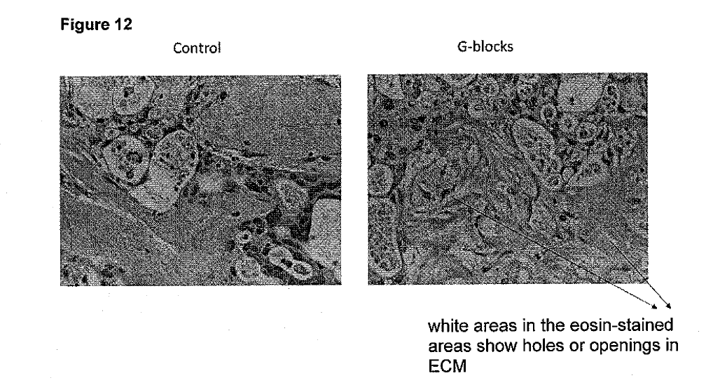

FIG. 11 shows the effect of G-blocks on tumour volume in a capan-2 human pancreatic tumour xenograft model. The dosing period in the experiment is indicated by the grey area. In each experiment, 5 groups of animals (n=10) were i.v. injected with vehicle, or G-block at three concentrations, corresponding to doses of 0.5 mg/kg, 25 mg/kg or 560 mg/kg.

FIG. 12 shows histological results. Tissue samples were formalin-fixed and embedded in paraffin, processed, and H & E stained. The white areas in the eosin-stained areas indicate holes or openings in the ECM.

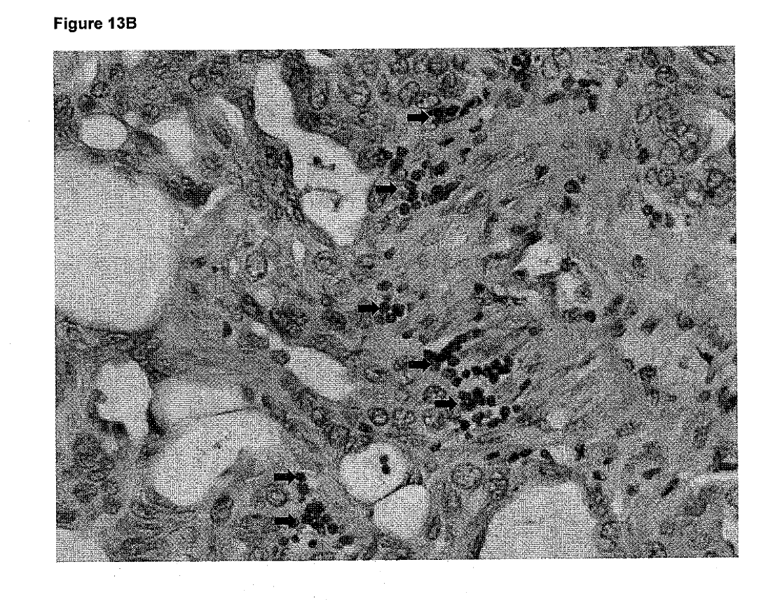

FIG. 13 shows histological results of the Example 2 tumour samples which were stained with H & E staining.

FIG. 14 shows time taken for total fluorescence recovery in Matrigel in a control and G-Block-treated sample.

### EXAMPLE 1

Effect of Oligouronates on Matrigel Properties

Matrigel (BD Biosciences) was used as received from the manufacturer. The tested G-block (dp 10, charcoal filtered) was solubilized at 10 mg/ml in physiological saline (150 mM NaCl). The G-block solution and physiological saline were chilled on ice and the Ependorf rack and pipette tips were chilled. The Matrigel (4× Ependorf containing 100 μl) was thawed on ice.

As a control, 100 μl physiological saline were added to each of 2 Matrigel Ependorfs, and pipetted up and down to achieve full mixing. The contents of the 2 tubes mixed and stored on ice.

To test the G-block, 100 μl G-block solution were added to each of 2 Matrigel Ependorfs, and pipetted up and down to achieve full mixing. The contents of the 2 tubes were mixed and stored on ice.

300 μl of the control or test Matrigel solution were applied to rheometer plates at 10° C. (using C 40/1 geometry) for rheological measurent Complex modulus (G* in Pa) was measured over a temperature sweep from 10-37° C., ½° minute⁻¹. G* is indicative of the mechanical properties of the material under test. The results are shown in FIG. 1.

The experiment was repeated including other oligouronates: control (as above), G-block10 (Dp 10, 94% G: 5 mg/ml), G-block20 (Dp 20, 90% G; 5 mg/mi), M-block (Dp 20, 100% M; 5 mg/ml) and galacturonate oligomer (not fully analysed but believed to have a Dp in the range 10-20 and more than 80% galacturonate (GaIU); 5 mg/ml). The results are shown in FIG. 2.

In the experiment only kinetics of gel formation have been examined and not equilibrium properties. Nonetheless, it can be seen from these Figures that oligouronates, particularly the alginate oligomers, and especially G-block oligomers (oligoguluronates) had an effect on the rheology of the matrigel ECM preparation, and in particular are effective in reducing G*. This clearly indicates that the structure is being affected (disrupted), and the solid-like behaviour of the ECM is reduced. In other words, oligoruronates reduce gelling or gel-like behaviour of the ECM.

### EXAMPLE 2

Effect of G Block Either Alone or in Combination with Gemcitabine in the Capan-2 Human Pancreas Tumour Xenograft Model

The purpose of this study was to evaluate the anti-tumour activity of G-Block as an enhancer in combination with gemcitabine in the Capan-2 human pancreas tumour xenograft model. Gemcitabine was dosed at a level that would not be curative and allow for proof of concept evaluation in this model. Efficacy was assessed by comparison of tumour weights of treated groups with the vehicle control group on Day 11 and 74 and between combination groups and their respective single agents on Day 11 and 74.

Materials and Methods

G-Block (Lot #PolyG 230712-Dp10, 95%G) was received as a white hygroscopic amorphous powder from NTNU Technology Transfer AS (Trondheim, Norway) and stored desiccated at room temperature until use. NTNU's G-Block was dissolved in MilliQ water to a concentration of 70 mg/ml to be used as the stock solution. This solution was used to deliver a dose of approximately 560 mg/kg in a 200 μl fixed dose. The 70 mg/ml solution was then diluted in 0.9% 150 mM NaCl (B. Braun Medical; Irvine, Calif.) to deliver doses of approximately 200 mg/kg, 75 mg/kg, 25 mg/kg, and 5 mg/kg in a 200 μL fixed dose volume. All dosing calculations assume a 25 g animal. NTNU's G-Block 70 mg/ml stock solution was formulated at the beginning of the study and used for dilutions before each dose, sterile filtered, and stored at 4° C. between doses. All unused dosing mixture was properly disposed of at TD2 after the study ended.

Gemcitabine (Lot #A892259C) was received as a solution from Eli Lilly and Co. (Indianapolis, Ind.) and stored at room temperature until use. Gemcitabine was diluted in saline (B. Braun Medical; Irvine, Calif.) to a concentration of 0.5 mg/ml to deliver a dosage of 5.0 mg/kg in a 10 ml/kg dose volume. Gemcitabine was formulated fresh prior to each dose. All unused dosing mixture was properly disposed of at TD2 after each dose.

Vehicle control was dosed with a solution of 0.9% 150 mM NaCl at a 10 ml/kg dose volume. Vehicle control was stored at room temperature.

Cell Culture

Capan-2 human pancreas tumour xenograft cell line was received from ATCC (Manassas, Va.). Cultures were maintained in McCoy's 5A medium (Hyclone, Logan, Utah) supplemented with 10% fetal bovine serum (Omega Scientific; Tarzana, Calif.), and housed in a 5% CO₂ atmosphere. The cultures were expanded in tissue culture flasks at a 1:5 split ratio until a sufficient amount of cells were harvested.

Animals

Female Athymic Nude mice (Hsd:Athymic Nude-Foxn1^{nu}) were supplied by Harlan (Germantown, N.Y.). Mice were received at 4 weeks of age. All mice were acclimated prior to handling. The mice were housed in microisolator cages (Lab Products, Seaford, Del.) and maintained under specific pathogen-free conditions. The mice were fed Tekland Global Diet® 2920× irradiated laboratory animal diet (Harlan, Indianapolis, Ind.) and autoclaved water was freely available. All procedures were carried out under the institutional guidelines of TGen Drug Development Institutional Animal Care and Use Committee (Protocol #13046).

### Cagan-2 Human Pancreas Tumor Xenograft Model

Female mice were inoculated subcutaneously in the right flank with 0.1 mL of a 50% RPMI/50% Matrigel™ (BD Biosciences, Bedford, Mass.) mixture containing a suspension of Capan-2 tumor cells (approximately 5.0×10⁶ cells/mouse). Seven days following inoculation, tumors were measured using calipers and tumor weight was calculated using the animal study management software, Study Director V.2.1.1 (Study Log)¹. Eighty mice with tumor sizes of 101-194 mg were randomized into eight groups of ten mice each by random equilibration with a mean of approximately 140 mg, using Study Director (Day 1). Body weights were recorded when the mice were randomized and were taken twice weekly thereafter in conjunction with tumor measurements. Dosing was performed as described below in Table 1.

TABLE 1
Evaluation of G-Block as a single agent and in combination with
gemcitabine in the Capan-2 human pancreatic tumour xenograft model

| | | Vehicle | G-Block* | Gemcitabine |
|---|---|---|---|---|
| Group | N | (Q3Dx4) | (Q3Dx4) | (Q3Dx4) |
| 1. Vehicle (IV) | 10 | X | | |
| 2. NTNU's G-Block | 10 | | X | |
| 560 mg/kg (IV) | | | | |
| 3. Gemcitabine | 10 | | | X |
| 5 mg/kg (IP) | | | | |
| 4. NTNU's G-Block | | | | |
| 560 mg/kg (IV) + | 10 | | X | X |
| Gemcitabine 5 mg/kg | | | | |
| (IP) | | | | |
| 5. NTNU's G-Block | 10 | | X | X |
| 200 mg/kg (IV) + | | | | |
| Gemcitabine 5 mg/kg | | | | |
| (IP) | | | | |
| 6. NTNU's G-Block | 10 | | X | X |
| 75 mg/kg (IV) + | | | | |
| Gemcitabine 5 mg/kg | | | | |
| (IP) | | | | |
| 7. NTNU's G-Block | 10 | | X | X |
| 25 mg/kg (IV) + | | | | |
| Gemcitabine 5 mg/kg | | | | |
| (IP) | | | | |
| 8. NTNU's G-Block | 10 | | X | X |
| 5 mg/kg (IV) + | | | | |
| Gemcitabine 5 mg/kg | | | | |
| (IP) | | | | |

*G-Block was dosed at 30 minutes post gemcitabine dose to align with gemcitabine's intraperitoneal Tmax.

All groups were terminated on Day 74. Mice were sacrificed prior to study end if tumor weight exceeded 1500 mg. Upon necropsy, tumors were excised from all animals in all groups and bisected in halves, placed in formalin vials (Azer Scientific/VWR; Franklin Lakes, N.J.) then fixed for approximately 24 hours before transferring to ethanol. Formalin-fixed tissues were embedded in paraffin, processed, and H & E stained.

Results

All treatment groups exhibited increasing body weights after the first few days of the study, indicating the treatment regimen was well-tolerated. Minor tumour necrosis was noted in some mice. One mouse (Group 2 Mouse 10) had non-typical clinical observations involving discoid papules and masses on the skin. This observation was not determined to be treatment related.

The results are summarised in FIGS. 3(A and B) and 4. As shown in the Figures, surprisingly, as a single agent G-block resulted in lower mean tumour weights compared to vehicle control at the end of the dosing phase. All combination treatments with G-block and gemcitabine resulted in significant decrease in mean tumour weight when compared to vehicle control at the end of the dosing phase. A decrease in mean tumour weight can be seen as compared with the single agents, and in particular gemcitabine alone.

### EXAMPLE 3

### Statistical Analysis

The following concerns a statistical analysis of tumour weights in the study reported in Example 2. This was a preclinical study in which 10 mice each were allocated to eight different treatments with Vehicle (placebo), G-Block 560 mg (enhancer), Gemcitabine 5 mg (oncology drug) or the combination of Gemcitabine 5 mg with G-Block 5 mg, 25 mg, 75 mg, 200 mg or 560 mg. Tumour weight and body weights were assessed Day 1 and then twice weekly up to last assessment on Day 74.

Statistical Analysis

Treatments were compared using an analysis of variance (ANOVA) model with fixed factor treatment and tumour weight at Day 1 as a covariate and assuming equal variance for each assessment (Day 1 model without covariate). Separate models were assigned to each day of assessment. Pairwise differences between active treatments and Vehicle was computed, as well as pairwise comparisons between G-Block 560 mg, Gemcitabine 5 mg and the combination of these two, output include 95% confidence interval for the difference and the associated p-value. Dose-response of G-Block on top of Gemcitabine 5 mg treatment was assessed by fitting a straight line to the treatment means from the basic ANOVA using weighted least squares regression. Areas under the curve (AUCs) were computed using the trapezoidal rule and further normalised by dividing with the interval length of integration, forming an average tumour weight (Eav). Eays were analysed using an ANOVA model with fixed factor treatment. Further tumour data was analysed on the log scale using a corresponding model as the main analysis on linear scale. This means that data was first logged, then analysed and finally the result transformed back to the original scale. Treatment means generated in this way will be geometric means and differences between treatments will be expressed as ratios of such geometric means. Further the standard deviation for the logged data can be transformed to a coefficient of variation (CV; relative standard deviation) for data on the original scale. Tumour weights of 0 mg (one mouse) were set to 10 mg in this analysis to avoid singularities. Data were complete except for 3 values during day 74. These has been kept as missing in the analysis, but when constructing mean value curves, imputed values based on last value extended were used instead.

Results

Tumour Weight—Analysis by Day

Individual curves of tumour weight by day and treatment are given in FIG. 5 and a corresponding mean value graph is given in FIG. 6. From a rather common starting tumour weight, some mice shows rapidly growing tumour weights whereas others are more or less unchanged, and this in all treatment groups. The resulting mean value curves therefore show an increasing trend with Vehicle treatment showing the fastest increase. Six (6) individual curves has been truncated at 2000 mg to keep a distinguishable scale (3 on Vehicle, 2 on Gemcitabine 5 mg and 1 on Gemcitabine 5 mg+G-Block 5 mg). Maximum tumour weight was 3321 mg. Three mice was terminated after Day 71 (one each on Vehicle, 2 on Gemcitabine 5 mg and 1 on Gemcitabine 5 mg+G-Block 5 mg) with tumour weights above 3000 mg. Table 2 summarises the results of a basal ANOVA analysis applied separately for each assessment day. Differences of active treatment versus Vehicle are illustrated in FIG. 7 (estimated treatment difference with 95% confidence intervals per day). Tumour weights were well balanced between treatments at Day 1. Treatment with all active agents, G-Block 560 mg, Gemcitabine 5 mg and combinations of Gemcitabine 5 mg and G-Block 5, 25, 75, 200 and 560 mg:s, reach a statistically significant difference versus Vehicle at Day 8. A consistent statistically significant effect can be shown with G-Block 560 mg up to Day 25, with Gemcitabine 5 mg up to Day 36, and for combinations of Gemcitabine and G-Block Day 43 (G-Block 5 and 560 mg), Day 46 (G-Block 25 and 200 mg) or Day 50 (G-Block 75 mg). The residual standard deviation in the models increase from 22 mg at Day 4 to 625 mg at Day 71. Treatment differences increases up to about 1 month after start of treatment and then remain rather constant in size, thus the loss of statistically significant effects is mainly an effect of the increasing variability in data.

TABLE 2

| | | Estimate | Difference vs Vehicle | | Residuai |
|---|---|---|---|---|---|
| Day | Treatment | (mg) | Diff. 95% conf. lim. P-value | | SD (mg) |
| Day 1 | Vehicle | 140.8 | | | 25.7 |
| | G-Block 560 | 140.5 | −0.3 | (−23.2-22.6) 0.981 | |
| | Gemcitabine 5 | 140.7 | −0.0 | (−23.0-22.9) 0.997 | |
| | Gem. 5 + G-Block 5 | 140.4 | −0.4 | (−23.3-22.5) 0.972 | |
| | Gem. 5 + G-Block 25 | 140.3 | −0.5 | (−23.4-22.5) 0.968 | |
| | Gem. 5 + G-Block 75 | 140.0 | −0.8 | (−23.7-22.1) 0.945 | |
| | Gem. 5 + G-Block 200 | 139.8 | −0.9 | (−23.9-22.0) 0.934 | |
| | Gem. 5 + G-Block 560 | 140.4 | −0.4 | (−23.3-22.6) 0.975 | |
| Day 4 | Vehicle | 175.6 | | | 21.8 |
| | G-Block 560 | 188.2 | 12.6 | (−6.9-32.1) 0.201 | |
| | Gemcitabine 5 | 189.8 | 14.1 | (−5.4-33.6) 0.153 | |
| | Gem. 5 + G-Block 5 | 157.2 | −18.4 | (−37.9-1.1) 0.064 | |
| | Gem. 5 + G-Block 25 | 161.2 | −14.4 | (−33.9-5.0) 0.144 | |
| | Gem. 5 + G-Block 75 | 181.0 | 5.4 | (−14.1-24.8) 0.584 | |
| | Gem. 5 + G-Block 200 | 178.9 | 3.3 | (−16.2-22.7) 0.739 | |
| | Gem. 5 + G-Block 560 | 166.9 | −8.7 | (−28.2-10.8) 0.375 | |
| Day 8 | Vehicle | 230.6 | | | 35.7 |
| | G-Block 560 | 184.6 | −46.0 | (−77.8-−14.1) 0.005 | |
| | Gemcitabine 5 | 161.6 | −69.0 | (−100.8-−37.1) <0.001 | |
| | Gem. 5 + G-Block 5 | 195.4 | −35.2 | (−67.0-−3.3) 0.031 | |
| | Gem. 5 + G-Block 25 | 188.7 | −41.9 | (−73.7-−10.0) 0.011 | |
| | Gem. 5 + G-Block 75 | 160.0 | −70.6 | (−102.5-−38.7) <0.001 | |
| | Gem. 5 + G-Block 200 | 142.7 | −87.9 | (−119.7-−56.0) <0.001 | |
| | Gem. 5 + G-Block 560 | 144.2 | −86.4 | (−118.3-−54.6) <0.001 | |
| Day 11 | Vehicle | 285.3 | | | 57.3 |
| | G-Block 560 | 205.0 | −80.3 | (−131.4-−29.1) 0.003 | |
| | Gemcitabine 5 | 174.2 | −111.0 | (−162.2-−59.9) <0.001 | |
| | Gem. 5 + G-Block 5 | 161.9 | −123.4 | (−174.5-−72.2) <0.001 | |
| | Gem. 5 + G-Block 25 | 165.1 | −120.2 | (−171.3-−69.0) <0.001 | |
| | Gem. 5 + G-Block 75 | 150.0 | −135.3 | (−186.4-−84.1) <0.001 | |
| | Gem. 5 + G-Block 200 | 144.8 | −140.4 | (−191.5-−89.3) <0.001 | |
| | Gem. 5 + G-Block 560 | 148.6 | −136.6 | (−187.8-−85.5) <0.001 | |
| Day 16 | Vehicle | 297.7 | | | 65.7 |
| | G-Block 560 | 199.2 | −98.6 | (−157.2-−39.9) 0.001 | |
| | Gemcitabine 5 | 181.4 | −116.3 | (−174.9-−57.7) <0.001 | |
| | Gem. 5 + G-Block 5 | 139.6 | −158.1 | (−216.7-−99.5) <0.001 | |
| | Gem. 5 + G-Block 25 | 186.3 | −111.4 | (−170.0-−52.8) <0.001 | |
| | Gem. 5 + G-Block 75 | 166.7 | −131.0 | (−189.7-−72.4) <0.001 | |
| | Gem. 5 + G-Block 200 | 156.7 | −141.1 | (−199.7-−82.4) <0.001 | |
| | Gem. 5 + G-Block 560 | 167.8 | −129.9 | (−188.6-−71.3) <0.001 | |
| Day 19 | Vehicle | 314.2 | | | 74.3 |
| | G-Block 560 | 204.2 | −110.0 | (−176.3-−43.7) 0.001 | |
| | Gemcitabine 5 | 194.7 | −119.5 | (−185.8-−53.2) 0.001 | |
| | Gem. 5 + G-Block 5 | 143.3 | −170.9 | (−237.2-−104.6) <0.001 | |
| | Gem. 5 + G-Block 25 | 198.0 | −116.2 | (−182.5-−49.9) 0.001 | |
| | Gem. 5 + G-Block 75 | 173.9 | −140.3 | (−206.6-−74.0) <0.001 | |
| | Gem. 5 + G-Block 200 | 167.4 | −146.8 | (−213.1-−80.5) <0.001 | |
| | Gem. 5 + G-Block 560 | 177.9 | −136.3 | (−202.6-−70.0) <0.001 | |
| Day 22 | Vehicle | 347.5 | | | 76.5 |
| | G-Block 560 | 247.6 | −99.9 | (−168.1-−31.7) 0.005 | |
| | Gemcitabine 5 | 225.0 | −122.5 | (−190.8-−54.3) 0.001 | |
| | Gem. 5 + G-Block 5 | 169.0 | −178.6 | (−246.8-−110.3) <0.001 | |
| | Gem. 5 + G-Block 25 | 195.0 | −152.5 | (−220.7-−84.3) <0.001 | |
| | Gem. 5 + G-Block 75 | 197.1 | −150.4 | (−218.7-−82.2) <0.001 | |
| | Gem. 5 + G-Block 200 | 196.0 | −151.6 | (−219.8-−83.3) <0.001 | |
| | Gem. 5 + G-Block 560 | 214.7 | −132.9 | (−201.1-−64.6) <0.001 | |
| Day 25 | Vehicle | 401.9 | | | 95.0 |
| | G-Block 560 | 286.8 | −115.1 | (−199.8-−30.3) 0.008 | |
| | Gemcitabine 5 | 244.0 | −157.9 | (−242.7-−73.1) <0.001 | |
| | Gem. 5 + G-Block 5 | 199.5 | −202.4 | (−287.1-−117.6) <0.001 | |
| | Gem. 5 + G-Block 25 | 200.1 | −201.8 | (−286.6-−117.1) <0.001 | |
| | Gem. 5 + G-Block 75 | 199.3 | −202.6 | (−287.3-−117.8) <0.001 | |
| | Gem. 5 + G-Block 200 | 214.9 | −187.0 | (−271.7-−102.2) <0.001 | |
| | Gem. 5 + G-Block 560 | 244.9 | −157.0 | (−241.7-−72.2) <0.001 | |
| Day 29 | Vehicle | 438.6 | | | 103.7 |
| | G-Block 560 | 346.8 | −91.8 | (−184.2-0.7) 0.052 | |
| | Gemcitabine 5 | 273.6 | −164.9 | (−257.4-−72.5) 0.001 | |
| | Gem. 5 + G-Block 5 | 214.5 | −224.1 | (−316.5-−131.7) <0.001 | |
| | Gem. 5 + G-Block 25 | 236.8 | −201.8 | (−294.2-−109.4) <0.001 | |
| | Gem. 5 + G-Block 75 | 224.6 | −214.0 | (−306.5-−121.6) <0.001 | |
| | Gem. 5 + G-Block 200 | 237.7 | −200.8 | (−293.3-−108.4) <0.001 | |
| | Gem. 5 + G-Block 560 | 276.6 | −162.0 | (−254.4-−69.6) 0.001 | |
| Day 32 | Vehicle | 518.6 | | | 142.8 |
| | G-Block 560 | 399.0 | −119.7 | (−247.0-7.6) 0.065 | |
| | Gemcitabine 5 | 310.1 | −208.5 | (−335.8-−81.2) 0.002 | |
| | Gem. 5 + G-Block 5 | 233.6 | −285.0 | (−412.3-−157.7) <0.001 | |
| | Gem. 5 + G-Block 25 | 276.5 | −242.1 | (−369.4-−114.8) <0.001 | |
| | Gem. 5 + G-Block 75 | 251.1 | −267.6 | (−394.9-−140.3) <0.001 | |
| | Gem. 5 + G-Block 200 | 252.4 | −266.2 | (−393.5-−138.9) <0.001 | |
| | Gem. 5 + G-Block 560 | 298.7 | −220.0 | (−347.3-−92.7) 0.001 | |
| Day 36 | Vehicle | 551.2 | | | 171.4 |
| | G-Block 560 | 431.9 | −119.3 | (−272.2-−33.6) 0.124 | |
| | Gemcitabine 5 | 360.9 | −190.3 | (−343.1-−37.4) 0.015 | |
| | Gem. 5 + G-Block 5 | 268.0 | −283.1 | (−436.0-−130.3) <0.001 | |
| | Gem. 5 + G-Block 25 | 301.7 | −249.5 | (−402.3-−96.6) 0.002 | |
| | Gem. 5 + G-Block 75 | 272.2 | −279.0 | (−431.9-−126.1) 0.001 | |
| | Gem. 5 + G-Block 200 | 268.2 | −283.0 | (−435.8-−130.1) <0.001 | |
| | Gem. 5 + G-Block 560 | 342.1 | −209.1 | (−362.0-−56.2) 0.008 | |
| Day 39 | Vehicle | 555.0 | | | 194.4 |
| | G-Block 560 | 454.6 | −100.4 | (−273.7-72.9) 0.252 | |
| | Gemcitabine 5 | 389.6 | −165.4 | (−338.7-8.0) 0.061 | |
| | Gem. 5 + G-Block 5 | 283.9 | −271.1 | (−444.4-−97.7) 0.003 | |
| | Gem. 5 + G-Block 25 | 318.8 | −236.1 | (−409.4-−62.8) 0.008 | |
| | Gem. 5 + G-Block 75 | 302.4 | −252.6 | (−425.9-−79.2) 0.005 | |
| | Gem. 5 + G-Block 200 | 289.5 | −265.5 | (−438.8-−92.1) 0.003 | |
| | Gem. 5 + G-Block 560 | 369.2 | −185.7 | (−359.1-−12.4) 0.036 | |
| Day 43 | Vehicle | 644.8 | | | 245.3 |
| | G-Block 560 | 493.8 | −150.9 | (−369.7-67.9) 0.173 | |
| | Gemcitabine 5 | 506.6 | −138.1 | (−356.9-80.6) 0.212 | |
| | Gem. 5 + G-Block 5 | 349.9 | −294.8 | (−513.6-−76.0) 0.009 | |
| | Gem. 5 + G-Block 25 | 374.5 | −270.3 | (−489.0-−51.5) 0.016 | |
| | Gem. 5 + G-Block 75 | 362.9 | −281.8 | (−500.6-−63.0) 0.012 | |
| | Gem. 5 + G-Block 200 | 359.2 | −285.5 | (−504.3-−66.8) 0.011 | |
| | Gem. 5 + G-Block 560 | 395.2 | −249.6 | (−468.4-−30.8) 0.026 | |
| Day 46 | Vehicle | 643.8 | | | 255.6 |
| | G-Block 560 | 562.9 | −81.0 | (−308.9-147.0) 0.481 | |
| | Gemcitabine 5 | 536.4 | −107.4 | (−335.3-120.5) 0.351 | |
| | Gem. 5 + G-Block 5 | 416.2 | −227.6 | (−455.5-0.3) 0.050 | |
| | Gem. 5 + G-Block 25 | 392.7 | −251.2 | (−479.1-−23.3) 0.031 | |
| | Gem. 5 + G-Block 75 | 392.0 | −251.9 | (−479.8-−23.9) 0.031 | |
| | Gem. 5 + G-Block 200 | 402.7 | −241.1 | (−469.0-−13.2) 0.038 | |
| | Gem. 5 + G-Block 560 | 443.5 | −200.3 | (−428.2-−27.6) 0.084 | |
| Day 50 | Vehicle | 703.9 | | | 289.4 |
| | G-Block 560 | 618.9 | −85.0 | (−343.0-173.0) 0.513 | |
| | Gemcitabine 5 | 573.5 | −130.4 | (−388.4-127.6) 0.317 | |
| | Gem. 5 + G-Block 5 | 457.3 | −246.6 | (−504.6-11.4) 0.061 | |
| | Gem. 5 + G-Block 25 | 450.9 | −253.0 | (−511.0-5.0) 0.054 | |
| | Gem. 5 + G-Block 75 | 409.7 | −294.2 | (−552.2-−36.2) 0.026 | |
| | Gem. 5 + G-Block 200 | 450.8 | −253.1 | (−511.2-4.9) 0.054 | |
| | Gem. 5 + G-Block 560 | 509.2 | −194.7 | (−452.7-−63.3) 0.137 | |
| Day 53 | Vehicle | 714.1 | | | 333.7 |
| | G-Block 560 | 639.2 | −74.9 | (−372.5-222.7) 0.617 | |
| | Gemcitabine 5 | 653.0 | −61.2 | (−358.8-236.4) 0.683 | |
| | Gem. 5 + G-Block 5 | 493.6 | −220.5 | (−518.1-−77.1) 0.144 | |
| | Gem. 5 + G-Block 25 | 487.2 | −227.0 | (−524.6-70.6) 0.133 | |
| | Gem. 5 + G-Block 75 | 448.7 | −265.5 | (−563.1-32.1) 0.080 | |
| | Gem. 5 + G-Block 200 | 477.7 | −236.4 | (−534.0-−61.2) 0.118 | |
| | Gem. 5 + G-Block 560 | 502.2 | −211.9 | (−509.5-−85.7) 0.160 | |
| Day 57 | Vehicle | 770.9 | | | 360.0 |
| | G-Block 560 | 693.3 | −77.6 | (−398.5-243.4) 0.631 | |
| | Gemcitabine 5 | 626.6 | −144.3 | (−465.3-176.6) 0.373 | |
| | Gem. 5 + G-Block 5 | 571.7 | −199.2 | (−520.2-121.7) 0.220 | |
| | Gem. 5 + G-Block 25 | 530.5 | −240.4 | (−561.4-80.6) 0.140 | |
| | Gem. 5 + G-Block 75 | 512.1 | −258.8 | (−579.8-62.2) 0.112 | |
| | Gem. 5 + G-Block 200 | 486.3 | −284.6 | (−605.6-36.4) 0.081 | |
| | Gem. 5 + G-Block 560 | 549.3 | −221.6 | (−542.6-−99.4) 0.173 | |
| Day 60 | Vehicle | 765.0 | | | 389.2 |
| | G-Block 560 | 743.1 | −21.9 | (−368.9-325.2) 0.900 | |
| | Gemcitabine 5 | 680.5 | −84.5 | (−431.5-262.6) 0.629 | |
| | Gem. 5 + G-Block 5 | 629.2 | −135.7 | (−482.8-211.3) 0.438 | |
| | Gem. 5 + G-Block 25 | 579.3 | −185.7 | (−532.7-161.4) 0.290 | |
| | Gem. 5 + G-Block 75 | 541.1 | −223.8 | (−570.9-123.3) 0.203 | |
| | Gem. 5 + G-Block 200 | 544.7 | −220.3 | (−567.3-126.8) 0.210 | |
| | Gem. 5 + G-Block 560 | 610.5 | −154.5 | (−501.6-192.6) 0.378 | |
| Day 64 | Vehicle | 793.4 | | | 458.5 |
| | G-Block 560 | 815.2 | 21.8 | (−387.1-430.7) 0.916 | |
| | Gemcitabine 5 | 869.5 | 76.1 | (−332.8-485.0) 0.712 | |
| | Gem. 5 + G-Block 5 | 719.1 | −74.4 | (−483.3-334.5) 0.718 | |
| | Gem. 5 + G-Block 25 | 643.6 | −149.8 | (−558.7-259.1) 0.467 | |
| | Gem. 5 + G-Block 75 | 605.0 | −188.4 | (−597.3-220.5) 0.361 | |
| | Gem. 5 + G-Block 200 | 620.8 | −172.6 | (−581.5-236.3) 0.403 | |
| | Gem. 5 + G-Block 560 | 702.1 | −91.4 | (−500.3-317.5) 0.657 | |
| Day 67 | Vehicle | 867.7 | | | 500.4 |
| | G-Block 560 | 897.6 | 29.9 | (−416.3-476.1) 0.894 | |
| | Gemcitabine 5 | 952.8 | 85.1 | (−361.1-531.3) 0.705 | |
| | Gem. 5 + G-Block 5 | 807.2 | −60.6 | (−506.8-385.6) 0.787 | |
| | Gem. 5 + G-Block 25 | 690.6 | −177.1 | (−623.3-269.1) 0.431 | |
| | Gem. 5 + G-Block 75 | 698.3 | −169.4 | (−615.6-276.8) 0.452 | |
| | Gem. 5 + G-Block 200 | 683.2 | −184.5 | (−630.8-261.7) 0.412 | |
| | Gem. 5 + G-Block 560 | 774.0 | −93.8 | (−540.0-352.4) 0.676 | |
| Day 71 | Vehicle | 1024.3 | | | 625.5 |
| | G-Block 560 | 925.3 | −99.1 | (−656.9-458.7) 0.724 | |
| | Gemcitabine 5 | 1078.5 | 54.2 | (−503.6-612.0) 0.847 | |
| | Gem. 5 + G-Block 5 | 936.1 | −88.2 | (−646.1-469.6) 0.753 | |
| | Gem. 5 + G-Block 25 | 850.7 | −173.6 | (−731.4-384.2) 0.537 | |
| | Gem. 5 + G-Block 75 | 738.3 | −286.0 | (−843.8-271.8) 0.310 | |
| | Gem. 5 + G-Block 200 | 749.5 | −274.8 | (−832.7-283.0) 0.329 | |
| | Gem. 5 + G-Block 560 | 797.5 | −226.8 | (−784.6-331.0) 0.420 | |
| Day 74 | Vehicle | 851.9 | | | 539.3 |
| | G-Block 560 | 994.4 | 142.6 | (−352.0-637.1) 0.567 | |
| | Gemcitabine 5 | 1009.1 | 157.2 | (−350.4-664.8) 0.539 | |
| | Gem. 5 + G-Block 5 | 1058.7 | 206.8 | (−287.8-701.3) 0.407 | |
| | Gem. 5 + G-Block 25 | 711.1 | −140.8 | (−648.1-366.6) 0.582 | |
| | Gem. 5 + G-Block 75 | 842.6 | −9.2 | (−503.7-485.2) 0.970 | |
| | Gem. 5 + G-Block 200 | 813.4 | −38.5 | (−532.9-456.0) 0.877 | |
| | Gem. 5 + G-Block 560 | 898.1 | 46.2 | (−448.3-540.7) 0.853 | |

Analysis of AUC

To get a combined measure over time, the area under the curve (AUC) for tumor weight was calculated. Different AUCs were calculated over the time intervals 0-60 days, 0-50 days, 0-39 days and 0-29 days. Values were further expressed as average tumor weight (Eav) over the intervals by dividing the AUC with the interval length.

Table 3 summarises the results of a basal ANOVA analysis for each Eav parameter computed. Statistically significant effects versus Vehicle could be shown for the combinations (all G-Block doses) for all intervals up to 60 days, for Gemcitabine 5 mg for intervals up to 50 days and for G-Block for intervals up to 39 days. As was seen when analysing individual days, the residual standard deviation increases with increasing length of the day interval included.

TABLE 3

| | | Estimate | Difference vs Vehicle | | Residuai |
|---|---|---|---|---|---|
| Parameter | Treatment | (mg) | Diff. 95% conf. lim. P-value | | SD (mg) |
| Eₐu, 0-60 | Vehicle | 468.0 | | | 151.5 |
| | G-Block 560 | 383.3 | −84.8 | (−219.8-50.3) 0.215 | |
| | Gemcitabine 5 | 349.7 | −118.4 | (−253.4-16.7) 0.085 | |
| | Gem. 5 + G-Block 5 | 281.3 | −186.7 | (−321.7-−51.7) 0.007 | |
| | Gem. 5 + G-Block 25 | 291.9 | −176.1 | (−311.2-−41.1) 0.011 | |
| | Gem. 5 + G-Block 75 | 274.9 | −193.1 | (−328.2-−58.1) 0.006 | |
| | Gem. 5 + G-Block 200 | 275.9 | −192.1 | (−327.1-−57.1) 0.006 | |
| | Gem. 5 + G-Block 560 | 309.3 | −158.7 | (−293.8-−23.7) 0.022 | |
| Eₐu, 0-50 | Vehicle | 413.3 | | | 116.3 |
| | G-Block 560 | 325.7 | −87.6 | (−191.3-16.0) 0.096 | |
| | Gemcitabine 5 | 292.1 | −121.2 | (−224.8-−17.5) 0.023 | |
| | Gem. 5 + G-Block 5 | 230.4 | −182.9 | (−286.5-−79.2) 0.001 | |
| | Gem. 5 + G-Block 25 | 248.2 | −165.1 | (−268.8-−61.5) 0.002 | |
| | Gem. 5 + G-Block 75 | 234.4 | −178.9 | (−282.6-−75.3) 0.001 | |
| | Gem. 5 + G-Block 200 | 234.2 | −179.1 | (−282.8-−75.5) 0.001 | |
| | Gem. 5 + G-Block 560 | 263.9 | −149.4 | (−253.0-−45.7) 0.005 | |
| Eₐu, 0-39 | Vehicle | 349.4 | | | 83.8 |
| | G-Block 560 | 267.6 | −81.8 | (−156.5-−7.1) 0.032 | |
| | Gemcitabine 5 | 231.3 | −118.1 | (−192.8-−43.4) 0.002 | |
| | Gem. 5 + G-Block 5 | 188.6 | −160.7 | (−235.5-−86.0) <0.001 | |
| | Gem. 5 + G-Block 25 | 209.9 | −139.5 | (−214.2-−64.8) <0.001 | |
| | Gem. 5 + G-Block 75 | 196.5 | −152.9 | (−227.6-−78.2) <0.001 | |
| | Gem. 5 + G-Block 200 | 194.3 | −155.1 | (−229.8-−80.4) <0.001 | |
| | Gem. 5 + G-Block 560 | 218.0 | −131.3 | (−206.1-−56.6) 0.001 | |
| Eₐu, 0-29 | Vehicle | 289.1 | | | 61.4 |
| | G-Block 560 | 218.0 | −71.1 | (−125.8-−16.3) 0.012 | |
| | Gemcitabine 5 | 195.5 | −93.6 | (−148.3-−38.8) 0.001 | |
| | Gem. 5 + G-Block 5 | 167.4 | −121.7 | (−176.5-−67.0) <0.001 | |
| | Gem. 5 + G-Block 25 | 183.8 | −105.3 | (−160.1-−50.6) <0.001 | |
| | Gem. 5 + G-Block 75 | 174.1 | −114.9 | (−169.7-−60.2) <0.001 | |
| | Gem. 5 + G-Block 200 | 171.5 | −117.6 | (−172.3-−62.8) <0.001 | |
| | Gem. 5 + G-Block 560 | 182.4 | −106.6 | (−161.4-−51.9) <0.001 | |
| Eₐu, 0-19 | Vehicle | 241.0 | | | 47.9 |
| | G-Block 560 | 188.1 | −52.9 | (−95.6-−10.2) 0.016 | |
| | Gemcitabine 5 | 173.4 | −67.6 | (−110.3-−24.9) 0.002 | |
| | Gem. 5 + G-Block 5 | 158.1 | −82.9 | (−125.6-−40.2) <0.001 | |
| | Gem. 5 + G-Block 25 | 172.3 | −68.7 | (−111.4-−26.0) 0.002 | |
| | Gem. 5 + G-Block 75 | 160.9 | −80.2 | (−122.8-−37.5) <0.001 | |
| | Gem. 5 + G-Block 200 | 153.4 | −87.6 | (−130.3-−44.9) <0.001 | |
| | Gem. 5 + G-Block 560 | 156.3 | −84.8 | (−127.5-−42.1) <0.001 | |

Multiplicative Analysis

With time, the tumor weights increases and so does the residual variability in the ANOVA models. Table 4 summarises the results of corresponding ANOVA models based on logged tumor weight data in an attempt to stabilise the variance. These multiplicative models gives geometric means as estimates and differences between treatment groups will be expressed as ratios of such geometric means. Estimated ratios versus Vehicle with 95% confidence intervals are illustrated in FIG. 8. The variability, expressed as CV, still shows an increasing trend, so the transformation (log-arithmation) has not been able to stabilise it fully. Regarding statistically significant effects (versus Vehicle) the results are very similar to the corresponding additive analyses results.

TABLE 4

| | | Estimate | Ratio vs Vehicle | | Residuai |
|---|---|---|---|---|---|
| Day | Treatment | (mg) | Ratio 95% conf. lim. P-value | | CV (%) |
| Day 1 | Vehicle | 138.5 | | | 18.2 |
| | G-Block 560 | 138.2 | 1.00 | (G.S5-1.17) 0.980 | |
| | Gemcitabine 5 | 138.6 | 1.00 | (G.S5-1.17) 0.998 | |
| | Gem. 5 + G-Block 5 | 138.6 | 1.00 | (G.S5-1.17) 0.994 | |
| | Gem. 5 + G-Block 25 | 138.5 | 1.00 | (G.S5-1.17) 0.997 | |
| | Gem. 5 + G-Block 75 | 138.0 | 1.00 | (G.S5-1.17) 0.961 | |
| | Gem. 5 + G-Block 200 | 137.8 | 1.00 | (G.S5-1.17) 0.951 | |
| | Gem. 5 + G-Block 560 | 138.4 | 1.00 | (0.85-1.17) 0.991 | |
| Day 4 | Vehicle | 172.4 | | | 12.2 |
| | G-Block 560 | 183.6 | 1.07 | (0.96-1.19) 0.251 | |
| | Gemcitabine 5 | 186.7 | 1.08 | (G.97-1.21) 0.147 | |
| | Gem. 5 + G-Block 5 | 154.7 | 0.90 | (G.SG-1.00) 0.051 | |
| | Gem. 5 + G-Block 25 | 159.8 | 0.93 | (0.S3-1.03) 0.171 | |
| | Gem. 5 + G-Block 75 | 179.7 | 1.04 | (0.94-1.16) 0.448 | |
| | Gem. 5 + G-Block 200 | 175.0 | 1.01 | (0.91-1.13) 0.786 | |
| | Gem. 5 + G-Block 560 | 164.6 | 0.95 | (0.86-1.06) 0.400 | |
| Day 8 | Vehicle | 220.0 | | | 17.8 |
| | G-Block 560 | 179.8 | 0.82 | (0.70-0.96) 0.013 | |
| | Gemcitabine 5 | 157.0 | 0.71 | (0.61-0.84) <0.001 | |
| | Gem. 5 + G-Block 5 | 191.1 | 0.87 | (0.74-1.02) 0.080 | |
| | Gem. 5 + G-Block 25 | 183.2 | 0.83 | (0.71-0.97) 0.023 | |
| | Gem. 5 + G-Block 75 | 158.7 | 0.72 | (0.62-0.84) <0.001 | |
| | Gem. 5 + G-Block 200 | 137.6 | 0.63 | (0.53-0.73) <0.001 | |
| | Gem. 5 + G-Block 560 | 141.7 | 0.64 | (0.55-0.75) <0.001 | |
| Day 11 | Vehicle | 261.4 | | | 21.8 |
| | G-Block 560 | 200.6 | 0.77 | (0.63-0.93) 0.008 | |
| | Gemcitabine 5 | 167.8 | 0.64 | (0.53-0.78) <0.001 | |
| | Gem. 5 + G-Block 5 | 156.7 | 0.60 | (0.49-0.73) <0.001 | |
| | Gem. 5 + G-Block 25 | 160.5 | 0.61 | (0.51-0.74) <0.001 | |
| | Gem. 5 + G-Block 75 | 145.9 | 0.56 | (0.46-0.68) <0.001 | |
| | Gem. 5 + G-Block 200 | 139.6 | 0.53 | (0.44-0.65) <0.001 | |
| | Gem. 5 + G-Block 560 | 146.4 | 0.56 | (0.46-0.68) <0.001 | |
| Day 16 | Vehicle | 275.4 | | | 26.2 |
| | G-Block 560 | 193.0 | 0.70 | (0.56-0.88) 0.003 | |
| | Gemcitabine 5 | 172.5 | 0.63 | (0.50-0.79) <0.001 | |
| | Gem. 5 + G-Block 5 | 136.3 | 0.49 | (0.39-0.62) <0.001 | |
| | Gem. 5 + G-Block 25 | 176.9 | 0.64 | (0.51-0.81) <0.001 | |
| | Gem. 5 + G-Block 75 | 158.6 | 0.58 | (0.46-0.72) <0.001 | |
| | Gem. 5 + G-Block 200 | 152.9 | 0.56 | (0.44-0.70) <0.001 | |
| | Gem. 5 + G-Block 560 | 162.6 | 0.59 | (0.47-0.74) <0.001 | |
| Day 19 | Vehicle | 288.4 | | | 27.6 |
| | G-Block 560 | 196.8 | 0.68 | (0.54-0.87) 0.002 | |
| | Gemcitabine 5 | 185.1 | 0.64 | (0.50-0.82) <0.001 | |
| | Gem. 5 + G-Block 5 | 140.3 | 0.49 | (0.38-0.62) <0.001 | |
| | Gem. 5 + G-Block 25 | 187.3 | 0.65 | (0.51-0.83) 0.001 | |
| | Gem. 5 + G-Block 75 | 164.1 | 0.57 | (0.45-0.72) <0.001 | |
| | Gem. 5 + G-Block 200 | 163.4 | 0.57 | (0.45-0.72) <0.001 | |
| | Gem. 5 + G-Block 560 | 172.9 | 0.60 | (0.47-0.76) <0.001 | |
| Day 22 | Vehicle | 321.7 | | | 25.6 |
| | G-Block 560 | 238.0 | 0.74 | (0.59-0.93) 0.009 | |
| | Gemcitabine 5 | 216.1 | 0.67 | (0.54-0.84) 0.001 | |
| | Gem. 5 + G-Block 5 | 166.5 | 0.52 | (0.41-0.65) <0.001 | |
| | Gem. 5 + G-Block 25 | 188.3 | 0.59 | (0.47-0.73) <0.001 | |
| | Gem. 5 + G-Block 75 | 187.7 | 0.58 | (0.47-0.73) <0.001 | |
| | Gem. 5 + G-Block 200 | 190.2 | 0.59 | (0.47-0.74) <0.001 | |
| | Gem. 5 + G-Block 560 | 211.8 | 0.66 | (0.53-0.82) <0.001 | |
| Day 25 | Vehicle | 363.2 | | | 27.2 |
| | G-Block 560 | 280.0 | 0.77 | (0.61-0.98) 0.032 | |
| | Gemcitabine 5 | 233.4 | 0.64 | (0.51-0.82) <0.001 | |
| | Gem. 5 + G-Block 5 | 197.2 | 0.54 | (0.43-0.69) <0.001 | |
| | Gem. 5 + G-Block 25 | 185.7 | 0.51 | (0.40-0.65) <0.001 | |
| | Gem. 5 + G-Block 75 | 188.8 | 0.52 | (0.41-0.66) <0.001 | |
| | Gem. 5 + G-Block 200 | 209.2 | 0.58 | (0.45-0.73) <0.001 | |
| | Gem. 5 + G-Block 560 | 239.9 | 0.66 | (0.52-0.84) 0.001 | |
| Day 29 | Vehicle | 403.5 | | | 30.9 |
| | G-Block 560 | 335.7 | 0.83 | (0.64-1.09) 0.178 | |
| | Gemcitabine 5 | 258.9 | 0.64 | (0.49-0.84) 0.002 | |
| | Gem. 5 + G-Block 5 | 210.6 | 0.52 | (0.40-0.68) <0.001 | |
| | Gem. 5 + G-Block 25 | 222.2 | 0.55 | (0.42-0.72) <0.001 | |
| | Gem. 5 + G-Block 75 | 203.8 | 0.51 | (0.39-0.66) <0.001 | |
| | Gem. 5 + G-Block 200 | 231.2 | 0.57 | (0.44-0.75) <0.001 | |
| | Gem. 5 + G-Block 560 | 270.4 | 0.67 | (0.51-0.88) 0.004 | |
| Day 32 | Vehicle | 460.9 | | | 37.1 |
| | G-Block 560 | 384.8 | 0.83 | (0.61-1.15) 0.265 | |
| | Gemcitabine 5 | 286.5 | 0.62 | (0.45-0.86) 0.004 | |
| | Gem. 5 + G-Block 5 | 228.4 | 0.50 | (0.36-0.68) <0.001 | |
| | Gem. 5 + G-Block 25 | 253.1 | 0.55 | (0.40-0.76) <0.001 | |
| | Gem. 5 + G-Block 75 | 220.0 | 0.48 | (0.35-0.66) <0.001 | |
| | Gem. 5 + G-Block 200 | 241.3 | 0.52 | (0.38-0.72) <0.001 | |
| | Gem. 5 + G-Block 560 | 291.0 | 0.63 | (0.46-0.87) 0.005 | |
| Day 36 | Vehicle | 478.2 | | | 44.8 |
| | G-Block 560 | 416.0 | 0.87 | (0.59-1.27) 0.469 | |
| | Gemcitabine 5 | 324.4 | 0.68 | (0.46-0.99) 0.046 | |
| | Gem. 5 + G-Block 5 | 260.5 | 0.54 | (0.37-0.80) 0.002 | |
| | Gem. 5 + G-Block 25 | 274.6 | 0.57 | (0.39-0.84) 0.005 | |
| | Gem. 5 + G-Block 75 | 225.5 | 0.47 | (0.32-0.69) <0.001 | |
| | Gem. 5 + G-Block 200 | 252.2 | 0.53 | (0.36-0.77) 0.001 | |
| | Gem. 5 + G-Block 560 | 333.4 | 0.70 | (0.48-1.02) 0.064 | |
| Day 39 | Vehicle | 451.4 | | | 55.0 |
| | G-Block 560 | 435.3 | 0.96 | (0.61-1.53) 0.875 | |
| | Gemcitabine 5 | 349.9 | 0.78 | (0.49-1.23) 0.272 | |
| | Gem. 5 + G-Block 5 | 276.3 | 0.61 | (0.39-0.97) 0.036 | |
| | Gem. 5 + G-Block 25 | 279.2 | 0.62 | (0.39-0.98) 0.040 | |
| | Gem. 5 + G-Block 75 | 238.4 | 0.53 | (0.33-0.84) 0.007 | |
| | Gem. 5 + G-Block 200 | 268.8 | 0.60 | (0.38-0.94) 0.027 | |
| | Gem. 5 + G-Block 560 | 358.8 | 0.79 | (0.50-1.26) 0.322 | |
| Day 43 | Vehicle | 520.3 | | | 61.3 |
| | G-Block 560 | 461.6 | 0.89 | (0.54-1.47) 0.637 | |
| | Gemcitabine 5 | 439.5 | 0.84 | (0.51-1.40) 0.506 | |
| | Gem. 5 + G-Block 5 | 340.7 | 0.65 | (0.40-1.08) 0.098 | |
| | Gem. 5 + G-Block 25 | 309.0 | 0.59 | (0.36-0.98) 0.043 | |
| | Gem. 5 + G-Block 75 | 279.4 | 0.54 | (0.32-0.89) 0.016 | |
| | Gem. 5 + G-Block 200 | 333.0 | 0.64 | (0.39-1.06) 0.082 | |
| | Gem. 5 + G-Block 560 | 384.9 | 0.74 | (0.45-1.22) 0.237 | |
| Day 46 | Vehicle | 534.0 | | | 65.0 |
| | G-Block 560 | 530.1 | 0.99 | (0.58-1.69) 0.978 | |
| | Gemcitabine 5 | 460.6 | 0.86 | (0.51-1.46) 0.580 | |
| | Gem. 5 + G-Block 5 | 405.5 | 0.76 | (0.45-1.29) 0.303 | |
| | Gem. 5 + G-Block 25 | 309.2 | 0.58 | (0.34-0.98) 0.043 | |
| | Gem. 5 + G-Block 75 | 287.4 | 0.54 | (0.32-0.91) 0.022 | |
| | Gem. 5 + G-Block 200 | 371.7 | 0.70 | (0.41-1.18) 0.177 | |
| | Gem. 5 + G-Block 560 | 430.9 | 0.81 | (0.48-1.37) 0.422 | |
| Day 50 | Vehicle | 570.6 | | | 70.4 |
| | G-Block 560 | 582.3 | 1.02 | (0.58-1.80) 0.944 | |
| | Gemcitabine 5 | 485.0 | 0.85 | (0.48-1.50) 0.568 | |
| | Gem. 5 + G-Block 5 | 447.2 | 0.78 | (0.45-1.38) 0.393 | |
| | Gem. 5 + G-Block 25 | 340.1 | 0.60 | (0.34-1.05) 0.072 | |
| | Gem. 5 + G-Block 75 | 285.3 | 0.50 | (0.28-0.88) 0.017 | |
| | Gem. 5 + G-Block 200 | 414.0 | 0.73 | (0.41-1.28) 0.262 | |
| | Gem. 5 + G-Block 560 | 497.1 | 0.87 | (0.49-1.53) 0.628 | |
| Day 53 | Vehicle | 573.1 | | | 82.9 |
| | G-Block 560 | 595.0 | 1.04 | (0.54-1.98) 0.908 | |
| | Gemcitabine 5 | 530.7 | 0.93 | (0.49-1.76) 0.813 | |
| | Gem. 5 + G-Block 5 | 483.6 | 0.84 | (0.44-1.61) 0.601 | |
| | Gem. 5 + G-Block 25 | 340.6 | 0.59 | (0.31-1.13) 0.112 | |
| | Gem. 5 + G-Block 75 | 280.1 | 0.49 | (0.26-0.93) 0.030 | |
| | Gem. 5 + G-Block 200 | 420.7 | 0.73 | (0.39-1.40) 0.343 | |
| | Gem. 5 + G-Block 560 | 483.9 | 0.84 | (0.44-1.61) 0.602 | |
| Day 57 | Vehicle | 575.5 | | | 90.9 |
| | G-Block 560 | 632.7 | 1.10 | (0.55-2.20) 0.786 | |
| | Gemcitabine 5 | 516.6 | 0.90 | (0.45-1.79) 0.757 | |
| | Gem. 5 + G-Block 5 | 552.8 | 0.96 | (0.48-1.92) 0.908 | |
| | Gem. 5 + G-Block 25 | 368.1 | 0.64 | (0.32-1.28) 0.202 | |
| | Gem. 5 + G-Block 75 | 312.5 | 0.54 | (0.27-1.09) 0.083 | |
| | Gem. 5 + G-Block 200 | 416.2 | 0.72 | (0.36-1.44) 0.353 | |
| | Gem. 5 + G-Block 560 | 536.8 | 0.93 | (0.47-1.86) 0.841 | |
| Day 60 | Vehicle | 540.1 | | | 96.3 |
| | G-Block 560 | 680.6 | 1.26 | (0.61-2.59) 0.525 | |
| | Gemcitabine 5 | 539.6 | 1.00 | (0.49-2.06) 0.998 | |
| | Gem. 5 + G-Block 5 | 609.4 | 1.13 | (0.55-2.32) 0.740 | |
| | Gem. 5 + G-Block 25 | 400.8 | 0.74 | (0.36-1.53) 0.413 | |
| | Gem. 5 + G-Block 75 | 324.9 | 0.60 | (0.29-1.24) 0.165 | |
| | Gem. 5 + G-Block 200 | 464.6 | 0.86 | (0.42-1.77) 0.679 | |
| | Gem. 5 + G-Block 560 | 595.1 | 1.10 | (0.54-2.27) 0.790 | |
| Day 64 | Vehicle | 556.8 | | | 98.2 |
| | G-Block 560 | 751.4 | 1.35 | (0.65-2.81) 0.417 | |
| | Gemcitabine 5 | 633.3 | 1.14 | (0.55-2.37) 0.727 | |
| | Gem. 5 + G-Block 5 | 685.6 | 1.23 | (0.59-2.56) 0.573 | |
| | Gem. 5 + G-Block 25 | 447.3 | 0.80 | (0.39-1.67) 0.553 | |
| | Gem. 5 + G-Block 75 | 364.7 | 0.65 | (0.31-1.36) 0.253 | |
| | Gem. 5 + G-Block 200 | 534.4 | 0.96 | (0.46-2.00) 0.911 | |
| | Gem. 5 + G-Block 560 | 686.9 | 1.23 | (0.59-2.57) 0.569 | |
| Day 67 | Vehicle | 608.4 | | | 101.4 |
| | G-Block 560 | 822.0 | 1.35 | (0.64-2.86) 0.426 | |
| | Gemcitabine 5 | 695.7 | 1.14 | (0.54-2.42) 0.723 | |
| | Gem. 5 + G-Block 5 | 768.7 | 1.26 | (0.60-2.67) 0.536 | |
| | Gem. 5 + G-Block 25 | 477.3 | 0.78 | (0.37-1.66) 0.520 | |
| | Gem. 5 + G-Block 75 | 411.7 | 0.68 | (0.32-1.43) 0.303 | |
| | Gem. 5 + G-Block 200 | 584.2 | 0.96 | (0.45-2.03) 0.914 | |
| | Gem. 5 + G-Block 560 | 754.4 | 1.24 | (0.59-2.62) 0.569 | |
| Day 71 | Vehicle | 653.6 | | | 111.2 |
| | G-Block 560 | 841.6 | 1.29 | (0.58-2.87) 0.531 | |
| | Gemcitabine 5 | 746.4 | 1.14 | (0.51-2.54) 0.742 | |
| | Gem. 5 + G-Block 5 | 894.3 | 1.37 | (0.61-3.04) 0.437 | |
| | Gem. 5 + G-Block 25 | 554.6 | 0.85 | (0.38-1.89) 0.683 | |
| | Gem. 5 + G-Block 75 | 414.1 | 0.63 | (0.28-1.41) 0.259 | |
| | Gem. 5 + G-Block 200 | 639.0 | 0.98 | (0.44-2.18) 0.955 | |
| | Gem. 5 + G-Block 560 | 777.3 | 1.19 | (0.53-2.65) 0.667 | |
| Day 74 | Vehicle | 562.6 | | | 106.3 |
| | G-Block 560 | 906.6 | 1.61 | (0.73-3.58) 0.236 | |
| | Gemcitabine 5 | 746.3 | 1.33 | (0.59-3.01) 0.493 | |
| | Gem. 5 + G-Block 5 | 1022.4 | 1.82 | (0.82-4.03) 0.140 | |
| | Gem. 5 + G-Block 25 | 528.0 | 0.94 | (0.41-2.13) 0.878 | |
| | Gem. 5 + G-Block 75 | 472.0 | 0.84 | (0.38-1.86) 0.662 | |
| | Gem. 5 + G-Block 200 | 701.9 | 1.25 | (0.56-2.77) 0.581 | |
| | Gem. 5 + G-Block 560 | 866.9 | 1.54 | (0.69-3.42) 0.283 | |

Model Based Approach

Studying the mean value curves in FIG. 6 an approximation with exponential functions seem to fit mean value data well. Thus a model,

y=C*exp(aⱼ*t)

with C being a constant (the common starting tumor weight), at the rate of tumor growth for treatment i and t the time in study (day number −1), was fitted to data. Only data up to 60 days were used in the modeling.

The resulting exponential curves are shown in FIG. 9 (black curves) together with the individual curves (thin red curves) and the observed mean value curves (blue curves). For all active treatments the two curve types are very similar. The Vehicle treatment shows some discrepancy though, since tumor weight on average here grows more than exponential in the beginning and then slows down. This is driven by the three mice with very rapid tumour growths seen in FIG. 9. On the other hand some mice given Vehicle shows almost no increase in tumor weight at all, so the exponential growths fits acceptable to represent an average behaviour in the group.

FIG. 10 shows the 8 fitted exponential curves. Due to their construction, the rates describes the differences between the treatments, and as a result differences will increase monotonously over time. Differences in rates between active treatments and Vehicle is given in Table 5. All active treatments had rates statistically significantly smaller than the Vehicle rate. Table 6 similarly shows differences in rates between active treatments. Rates were statistically significantly smaller for all combinations of G-Block and Gemcitabine versus both G-Block 560 mg and Gemcitabine 5 mg when administered as mono-treatments.

TABLE 5
Estimated differences vs Vehicle in growth rates

| Contrast | Estimate | 95% C.I. | p-value |
|---|---|---|---|
| G-Block 560 vs Vehicle | −0.0027 | (−0.0046-−0.0007) | 0.007 |
| Gemcitabine 5 vs Vehicle | −0.0042 | (−0.0062-−0.0022) | <0.001 |
| G-B 5 + Gem. 5 vs Vehicle | −0.0088 | (−0.0111-−0.0065) | <0.001 |
| G-B 25 + Gem. 5 vs Vehicle | −0.0086 | (−0.0109-−0.0063) | <0.001 |
| G-B 75 + Gem. 5 vs Vehicle | −0.0099 | (−0.0124-−0.0075) | <0.001 |
| G-B 200 + Gem. 5 vs Vehicle | −0.0097 | (−0.0121-−0.0072) | <0.001 |
| G-B 560 + Gem. 5 vs Vehicle | −0.0071 | (−0.0093-−0.0049) | <0.001 |

TABLE 6
Estimated differences between active treatments in growth rates

| Contrast | Estimate | 95% C.I. | p-value |
|---|---|---|---|
| Gemcitabine 5 vs G-B 560 | −0.0015 | (−0.0037-0.0006) | 0.166 |
| G-B 5 + Gem. 5 vs G-B 560 | −0.0061 | (−0.0085-−0.0037) | <0.001 |
| G-B 25 + Gem. 5 vs G-B 560 | −0.0059 | (−0.0084-−0.0035) | <0.001 |
| G-B 75 + Gem. 5 vs G-B 560 | −0.0072 | (−0.0098-−0.0047) | <0.001 |
| G-B 200 + Gem. 5 vs G-B | −0.0070 | (−0.0095-−0.0045) | <0.001 |
| 560 | | | |
| G-B 560 + Gem. 5 vs G-B | −0.0045 | (−0.0068-−0.0021) | <0.001 |
| 560 | | | |
| G-B 5 + Gem. 5 vs Gem. 5 | −0.0046 | (−0.0071-−0.0021) | <0.001 |
| G-B 25 + Gem. 5 vs Gem. 5 | −0.0044 | (−0.0070-−0.0019) | 0.001 |
| G-B 75 + Gem. 5 vs Gem. 5 | −0.0057 | (−0.0084-−0.0031) | <0.001 |
| G-B 200 + Gem. 5 vs Gem. 5 | −0.0055 | (−0.0081-−0.0029) | <0.001 |
| G-B 560 + Gem. 5 vs Gem. 5 | −0.0030 | (−0.0054-−0.0005) | 0.016 |

### EXAMPLE 4

Further Study of the Effect of G-Block Alone in the Capan-2 Human Pancreas Tumour Xenograft Model

The aim of this experiment was to investigate dose-response and compare different dosage regimes. Materials and methods were as described in Example 2 in respect of G-blocks alone. Two different dosing regimens of G-Block were tested. The first, Q3x4, involved injection of G-Block every third day with a total of four injections, i.e. as performed in Example 2. In the second regimen, Q3x10, injections of G-Block were given every third day, with a total of ten injections. The results are given in FIG. 11. In each experiment, 5 groups of animals (n=10) were i.v. injected with vehicle, or G-block at three concentrations, corresponding to doses of 0.5 mg/kg b.w., 25 mg/kg b.w. or 560 mg/kg b.w.

### EXAMPLE 5

Effect of G-Block on ECM Structure

The tumours from Example 2 were analysed by histological analysis after finishing the study. Upon necropsy, tumors were excised from all animals in all groups and bisected in halves, placed in formalin vials (Azer Scientific/VWR; Franklin Lakes, N.J.) then fixed for approximately 24 hours before transferring to ethanol. Formalin-fixed tissues were embedded in paraffin, processed, and H & E stained. The histology results in FIG. 12 show that animals treated with G-blocks showed less dense extracellular matrix, as demonstrated by higher percentage of white areas within the eosin-stained areas.

### EXAMPLE 6

Evaluation of Anti-Tumour Activity of G-Block as a Single Agent and in Combination with Herceptin in the SK-OV-3 Human Ovarian Tumour Xenograft Model

The purpose of this study is to evaluate the anti-tumour activity of G-Block as an enhancer in combination with Herceptin in the SK-OV-3 human ovarian tumour xenograft model.

G-Block (Lot #PolyG 230712-(Dp10, 95%G)) as a white hygroscopic amorphous powder is supplied from NTNU Technology Transfer AS (Trondheim, Norway) and stored desiccated at room temperature until use. NTNU's G-Block is dissolved in MilliQ water to a concentration of 70 mg/ml to be used as the stock solution. This solution is used to deliver a dose of approximately 25 g/kg bw in a 60 μl fixed dose. The 70 mg/ml solution is diluted in 0.9% 150 mM NaCl to prepare other dosing solutions of 25 mg/kg or 0.5 mg/kg in a 60 μL fixed dose volume. All dosing calculations assume a 25 g animal. NTNU's G-Block 70 mg/ml stock solution is formulated at the beginning of the study and used for dilutions before each dose, sterile filtered, and stored at 4° C. between doses. All unused dosing mixture is properly disposed of at TD2 after the study ended.

Trastuzumab (Herceptin) is stored at room temperature until use. Trastuzumab is administered at a dose of 10 mg/kg in a volume of 10 ml/kg.

Vehicle control is dosed with a solution of 0.9% 150 mM NaCl at a 60 μl fixed dose volume. Vehicle control is stored at room temperature.

Cell Culture

SK-OV-3 human ovarian tumour xenograft cell line is obtained from ATCC (Manassas, Va.). Cultures are maintained in medium supplemented with 10% foetal bovine serum, and housed in a 5% CO₂ atmosphere. The cultures were expanded in tissue culture flasks until a sufficient amount of cells were harvested.

Animals

Female Athymic Nude mice are supplied by Harlan (Germantown, N.Y.). Mice are received at between 5 and 8 weeks of age. The mice are maintained under specific pathogen-free conditions. The mice are fed Tekland Global Diet® 2920× irradiated laboratory animal diet (Harlan, Indianapolis, Ind.) and autoclaved water is freely available.

SK-OV-3 Human Ovarian Tumor Xenograft Model

Female mice are inoculated subcutaneously in the right flank with 0.1 mL of a 50% medium/50% Matrigel™ (BD Biosciences, Bedford, Mass.) mixture containing a suspension of SK-OB-3 tumour cells (approximately 5.0×10⁶ cells/mouse).

Treatment is initiated on day 1, according to Table 7 below. Tumour volume and body weight are measured twice weekly. Dosing is performed as described below in Table 7 below.

TABLE 7
Evaluation of G-Block as a single agent and in combination with
Herceptin in the SK-OV-3 human ovarian tumour xenograft model

| | | Vehicle | Herceptin | G-Block |
|---|---|---|---|---|
| Group | N | (Q3Dx4) | (Q3Dx4) | (Q3Dx4) |
| 1. Vehicle (IV) | 10 | X | | |
| 2. Herceptin 10 mg/kg (IV) | 10 | | X | |
| 3. NTNU's G-Block 25 mg/kg | 10 | | | X |
| (IV) | | | | |
| 4. NTNU's G-Block | 10 | | X | X |
| 0.5 mg/kg (IV) + | | | | |
| Herceptin 10 mg/kg | | | | |
| (IP) | | | | |
| 5. NTNU's G-Block 25 mg/kg | 10 | | X | X |
| (IV) + Herceptin 10 mg/kg | | | | |
| (IP) | | | | |

Mice are sacrificed prior to study end if tumor volume exceeds 3000 mm³. Upon necropsy, tumors are excised from all animals in all groups and bisected in halves, placed in formalin vials (Azer Scientific/VWR; Franklin Lakes, N.J.) and fixed for approximately 24 hours before transferring to ethanol. Formalin-fixed tissues are embedded in paraffin, processed, and H & E stained.

Statistical Analysis

Student t-test and % T/C calculations are performed. Growth curves and percent mouse weight change graphs are produced to evaluate dose tolerance of the therapies.

### EXAMPLE 7

Lymphocyte Infiltration of Tumour Tissue

The samples of tumour tissue as obtained in Example 2 were removed from mice post mortem, paraffin embedded and prepared for light microscopy with H&E staining.

Differences in the observation of lymphocytes within the tissue sections from mice in the control and G-block treated groups were observed (see FIGS. 13A and B). There was a greater degree of lymphocyte infiltration in the tumour tissue from the mice that had received G-block treatment compared to tumour tissue from mice in the control groups, which did not receive G-block treatment. Showing clusters of infiltrating lymphocytes (red arrows). The mice are athymic nude mice, which lack T lymphocytes (killer T-cells) but possess natural killer cells (NK cells).

### EXAMPLE 8

### IqG ALEXA488 labeled IqG Mobility in Matrigel as Determined by Fluorescence Recovery after Photobleaching

Extracellular matrix gels were prepared cold from thawed Matrigel (BD Biosciences) at 75% of the received concentration, containing 2 μg/ml ALEXA488IgG (goat anti human, Life technologies) and either 5 ml/ml G-block (DPn 12, 93% G) or ionic strength matched saline control). 200 μl samples were pipetted into coverglass chambers, sealed, and heated at 37° C. for 30 minutes to induce gelation. High intensity laser light (488 line of the argon laser) was used to bleach the fluorophore labelled IgG in a region of interest and the recovery of fluorescence in this region (as a result of unbleached labelled IgG diffusing into the region of interest from outside the bleach region) was monitored.

The time (in seconds) to achieve total fluorescence recovery for 20 measurements on control and G-block treated Matrigel is presented below (mean±S.D.). The results are shown in FIG. 14. The difference between groups is statistically significant by Student's t-test (p=0.0005).

1-28. (canceled)

29. A method of treatment or prevention of cancer in a subject comprising administering an anticancer oligouronate to said subject, either alone or in combination with another anticancer agent, wherein the oligouronate contains up to 100 monomer residues, at least 70% of which are guluronate residues.

30. The method of claim 29, wherein the oligouronate is a 2 to 75-mer, 2 to 70-mer, 2 to 50-mer, 2 to 35-mer, 2- to 30-mer, 3- to 35-mer, 3- to 28-mer, 4- to 25-mer, 6- to 22-mer, 8- to 20-mer or 10- to 15-mer.

31. The method of claim 29, wherein the number average degree of polymerization (DPn) of the oligouronate is 2 to 100, 2 to 75, 2 to 50, 2 to 35, or 2 to 30.

32. The method of claim 29, wherein the oligouronate has at least 75%, 80%, 85% or 90% of guluronate residues.

33. The method of claim 29, wherein the oligouronate is for systemic administration or for administration directed to a tumor.

34. The method of claim 29, wherein the oligouronate is for intravenous administration.

35. The method of claim 29, wherein said oligouronate is not complexed with or contained within a cationic polymeric material and/or is not contained in or present on a surface of a vesicle or particle and/or is not complexed with or contained within a form of a microparticle.

36. The method of claim 29, wherein the oligouronate is water-soluble.

37. The method of claim 29, wherein the cancer is one or more solid tumor(s).

38. The method of claim 29, wherein the cancer involves an altered extracellular matrix (ECM).

39. The method of claim 38, wherein the ECM is thickened and/or stiffer and/or denser as compared to a corresponding ECM in non-cancerous tissue.

40. The method of claim 38, wherein the cancer exhibits stromal involvement or desmoplastic tumor(s).

41. The method of claim 29, wherein the cancer is selected from the group consisting of breast cancer, ovarian cancer and pancreatic cancer.

42. The method of claim 29, wherein the subject is a human.

43. The method of claim 29, wherein the anticancer agent is a small molecule having a molecular weight of less than 2000 Da, or less than 1000 Da.

44. The method of claim 29, wherein the anticancer agent is a chemotherapy drug.

45. The method of claim 29, wherein the anticancer agent is an immunotherapy agent selected from the group consisting of an antibody, a cytokine and a checkpoint inhibitor.

46. The method of claim 45, wherein the antibody is conjugated or fused to a toxin or a radioactive compound.

47. The method of claim 45, wherein the cytokine is selected from the group consisting of interluekin-2 and interferon-α.

48. The method of claim 45, wherein the checkpoint inhibitor targets PD-L1 or CTLA4.

49. The method of claim 29, wherein the anticancer oligouronate enhances delivery of the other anticancer agent.

50. A method for enhancing a delivery of an anticancer agent, said method comprising co-administering said anticancer agent with an oligouronate to a subject in need of said anticancer agent, wherein the oligouronate contains up to 100 monomer residues, at least 70% of which are guluronate residues.

51. The method of claim 50, wherein the anticancer agent is a small molecule having a molecular weight of less than 2000 Da, or less than 1000 Da.

52. The method of claim 50, wherein the anticancer agent is an immunotherapy agent selected from the group consisting of an antibody, a cytokine and a checkpoint inhibitor.

53. A kit comprising an anticancer oligouronate, wherein the oligouronate contains up to 100 monomer residues, at least 70% of which are guluronate residues, and another anticancer agent for use in treatment or prevention of cancer, and/or for enhancing a delivery of an anticancer agent to a subject in need thereof.
